(19) United States
(12) Patent Application Publication
(10) Pub. No.: US 2023/0368867 A1
(43) Pub. Date: Nov. 16, 2023

(54) SYSTEMS AND METHODS FOR IDENTIFYING OPTIMAL D GENE ASSIGNMENT AND/ORJUNCTION REGION STRUCTURE

(71) Applicant: 10x Genomics, Inc., Pleasanton, CA (US)

(72) Inventors: David Benjamin Jaffe, Pleasanton, CA (US); Wyatt James McDonnell, Concord, CA (US)

(21) Appl. No.: 18/310,524

(22) Filed: May 1, 2023

Related U.S. Application Data
(60) Provisional application No. 63/337,510, filed on May 2, 2022.

(51) Int. Cl. G16B 30/10; G16B 15/30

(52) U.S. Cl. CPC G16B 30/10; G16B 15/30

(57) ABSTRACT

A method is provided for identifying one or more D gene segment in a VDJ or VDDJ sequence. The method can include obtaining a B cell receptor and/or T cell receptor data set, wherein the data set includes a VDJ sequence, aligning the VDJ sequence to one or more VDJ reference sequences thereby generating a first potential alignment and a second potential alignment, determining a first score for the first potential alignment and a second score for the second potential alignment in accordance with a D gene segment alignment scoring schema, and identifying a D gene segment region associated with a highest score between the first score and the second score.

### CROSS REFERENCE TO RELATED APPLICATION

The present application claims the benefit of priority under 35 U.S.C. § 119(e) to U.S. Provisional Application No. 63/337,510, SYSTEMS AND METHODS FOR IDENTIFYING OPTIMAL D GENE ASSIGNMENT AND/OR JUNCTION REGION STRUCTURE, filed on May 2, 2022, which is currently co-pending herewith and which is incorporated by reference in its entirety.

### BACKGROUND

The immune system recognizes and eliminates non-self threats through a complex and layered network of both innate and adaptive immune cells. Robust characterization of this response and characterization of VDJ sequences has proven challenging to perform in a high-throughput fashion.

Current analysis platforms purportedly assign D genes yet cannot assign them confidently. Moreover, D gene assignments are not guaranteed to be consistent across a clonotype. These assignments are made, even though they are not confident, as they generally allow one to better understand what happened during junction region rearrangement. However, given current limitations, that understanding is often incomplete. This weakness of assignment is a consequence, for example, of the biology: D genes are short, and junction regions can be heavily edited during somatic hypermutation (SHM) and through non-templated indels during V(D)J recombination. As such, it is currently possible that where a D gene is aligned to given transcript bases, it is not the right D gene, or that the transcript bases represent some other part of the genome (not a D gene at all), or even random bases that were created during formation of the junction region.

As such, there is a need for systems and methods that can more accurately determine optimal D gene assignment and/or junction region structure.

### SUMMARY

In accordance with various embodiments, a method for identifying one or more D gene segment in a VDJ or VDDJ sequence is provided. The method can include obtaining a B cell receptor and/or T cell receptor data set, wherein the data set includes a VDJ sequence. The method can also include aligning the VDJ sequence to one or more VDJ reference sequences thereby generating a first potential alignment and a second potential alignment. The method can also include determining a first score for the first potential alignment and a second score for the second potential alignment in accordance with a D gene segment alignment scoring schema. The method can further include identifying a D gene segment region associated with a highest score between the first score and the second score.

In accordance with various embodiments, a non-transitory computer-readable medium in which a program is stored for causing a computer to perform a method for identifying one or more D gene segment in a VDJ or VDDJ sequence is provided. The method can comprise obtaining a B cell receptor and/or T cell receptor data set, wherein the data set includes a VDJ sequence. The method can also include aligning the VDJ sequence to one or more VDJ reference sequences thereby generating a first potential alignment and a second potential alignment. The method can also include determining a first score for the first potential alignment and a second score for the second potential alignment in accordance with a D gene segment alignment scoring schema. The method can further include identifying a D gene segment region associated with a highest score between the first score and the second score.

In accordance with various embodiments, a system for identifying one or more D gene segment in a VDJ or VDDJ sequence is provided. The method can comprise a data source configured to obtain a B cell receptor and/or T cell receptor data set, wherein the data set includes a VDJ sequence. The method can further include a processing unit configured to receive the B cell receptor and/or T cell receptor data set from the data source. The processing unit can include an alignment engine configured to align the VDJ sequence to one or more VDJ reference sequences thereby generating a first potential alignment and a second potential alignment. The processing unit can also include a scoring engine configured to determine a first score for the first potential alignment and a second score for the second potential alignment in accordance with a D gene segment alignment scoring schema. The processing unit can further include an identification engine configured to identify a D gene segment region associated with a highest score between the first score and the second score.

In some embodiments, aligning the VDJ sequence to one or more VDJ reference sequences includes applying a first affine gap penalty function when aligning regions between VDJ segments of the VDJ sequence and a second affine gap penalty function when aligning other regions of the VDJ sequence. In some embodiments, first affine gap penalty function penalizes gap opens for insertion between VDJ segments at a first rate, and wherein the second affine gap penalty function penalizes gap opens for deletion bridging VDJ segments at a second rate, or penalizes other gap opens at a third rate that is larger than the first rate and the second rate, penalizes gap extends for insertion between VDJ segments at a fourth rate, and penalizes other gap extends at a fifth rate that is higher than the fourth rate.

In some embodiments, the methods further include: applying a pre-determined scoring adjustment factor to the score of the 1st and 2nd potential alignments of the D gene segment region for the VDJ sequence. In some embodiments, the methods further include: identifying the potential alignment with the highest score as a correct alignment of the D gene segment region.

In some embodiments, aligning includes determining a first alignment score and a second alignment score. In some embodiments, determining the first score includes adding 2.2 times a first bit score to the first alignment score, wherein:

$$bit score = {\sum }_{l = 0}^{k} ( \begin{matrix}n \\ l\end{matrix} ) * \frac{{3}^{l}}{{4}^{n}}$$

where n is the sequence length, and k is a number of mismatches.

In some embodiments, determining the second score includes adding 2.2 times a second bit score to the second alignment score, wherein:

$$bit score = {\sum }_{l = 0}^{k} ( \begin{matrix}n \\ l\end{matrix} ) * \frac{{3}^{l}}{{4}^{n}}$$

where n is the sequence length, and k is a number of mismatches.

In some embodiments, the methods further include identifying an additional D gene segment, which is present in a VDDJ sequence

These and other aspects and implementations are discussed in detail herein. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 is a schematic illustration of a non-limiting example workflow for grouping lymphoid cells within a lymphoid cell variable domain region sequence dataset, in accordance with various embodiments.

FIG. 2 is a flow chart illustrating a non-limiting example method for grouping lymphoid cells within a lymphoid cell variable domain region sequence dataset, in accordance with various embodiments.

FIG. 3 is a diagram illustrating a non-limiting example system for grouping lymphoid cells within a lymphoid cell variable domain region sequence dataset, in accordance with various embodiments.

FIG. 4 is a block diagram that illustrates a computer system, upon which embodiments, or portions of the embodiments, may be implemented, in accordance with various embodiments.

FIG. 5A is a diagram illustrating an overview of clonotyping, in accordance with various embodiments. FIG. 5B is a diagram illustrating a process of B-cell clonotyping, In accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

Unless defined otherwise, all technical and scientific terms used herein have the same meaning as commonly understood by one of ordinary skill in the art to which there various embodiments belong.

All publications mentioned herein are incorporated herein by reference for the purpose of describing and disclosing devices, compositions, formulations and methodologies which are described in the publication and which might be used in connection with the present disclosure.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina, the GRIDION and PROMETHION Systems of Oxford Nanopore Technologies, PACBIO SEQUEL Systems of Pacific Biosciences, and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.) which denotes a single or a grouping of genes (in DNA or RNA) that have undergone changes as referenced against a particular species or sub-populations within a particular species due to mutations, recombination/crossover or genetic drift.

The term “B cells”, also known as B lymphocytes, refer to a type of white blood cell of the small lymphocyte subtype. They function in the humoral immunity component of the adaptive immune system by secreting antibodies. Additionally, B cells present antigen (they are also classified as professional antigen-presenting cells (APCs)) and secrete cytokines. In mammals, B cells mature in the bone marrow, which is at the core of most bones. In birds, B cells mature in the bursa of Fabricius, a lymphoid organ where they were first discovered by Chang and Glick, (B for bursa) and not from bone marrow as commonly believed. B cells, unlike the other two classes of lymphocytes, T cells and natural killer cells, express B cell receptors (BCRs) on their cell membrane. BCRs allow the B cell to bind to a specific antigen, against which it will initiate an antibody response.

The term “T cell”, also known as T lymphocytes, refer to a type of an adaptive immune cell. T cells develops in the thymus gland, hence the name T cell, and play a central role in the immune response of the body. T cells can be distinguished from other lymphocytes by the presence of a T cell receptor (TCR) on the cell surface. These immune cells originate as precursor cells, derived from bone marrow, and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the T cell receptor chain, T cells can also include T cells that express αβ TCR chains, T cells that express γδ TCR chains, as well as unique TCR co-expressors (i.e., hybrid αβ-γδ T cells) that co-express the αβ and γδ TCR chains.

T cells can also include engineered T cells that can attack specific cancer cells. A patient's T cells can be collected and genetically engineered to produce chimeric antigen receptors (CAR). These engineered T cells are called CAR T cells, which forms the basis of the developing technology called CAR-T therapy. These engineered CAR T cells are grown by the billions in the laboratory and then infused into a patient's body, where the cells are designed to multiply and recognize the cancer cells that express the specific protein. This technology, also called adoptive cell transfer is emerging as a potential next-generation immunotherapy treatment.

T cells, such as the killer T cells can directly kill cells that have already been infected by a foreign invader. T cells can also use cytokines as messenger molecules to send chemical instructions to the rest of the immune system to ramp up its response. Activating T cells against cancer cells is the basis behind checkpoint inhibitors, a relatively new class of immunotherapy drugs that have recently been approved to treat lung cancer, melanoma, and other difficult cancers. Cancer cells often evade patrolling T cells by sending signals that make them seem harmless. Checkpoint inhibitors disrupt those signals and prompt the T cells to attack the cancer cells.

The term “naïve”, as used herein, can refer to B-lymphocytes or T-lymphocytes that have not yet reacted with an epitope of an antigen or that have a cellular phenotype consistent with that of a lymphocyte that has not yet responded to antigen-specific activation after clonal licensing.

The term “Fab”, also referred to as an antigen-binding fragment, refers to the variable portions of an antibody molecule with a paratope that enables the binding of a given epitope of a cognate antigen. The amino acid and nucleotide sequences of the Fab portion of antibody molecules are hypervariable. This is in contrast to the “Fc” or crystallizable fragment, which is relatively constant and encodes the isotype for a given antibody; this region can also confer additional functional capacity through processes such as antibody-dependent complement deposition, cellular cytotoxicity, cellular trogocytosis, and cellular phagocytosis.

The phrase “clonal selection” refers to the selection and activation of specific B lymphocytes and T lymphocytes by the binding of epitopes to B cell receptors or T cell receptors with a corresponding fit and the subsequent elimination (negative selection) or licensing for clonal expansion (positive selection) of a B or T lymphocyte after binding of an antigenic determinant.

The phrase “clonal expansion” refers to the proliferation of B lymphocytes and T lymphocytes activated by clonal selection in order to produce a clonal population of daughter cells with the same antigen specificity and functional capacity. In the case of T lymphocytes this antigen specificity is exact at the nucleotide and protein level and in the case of B lymphocytes this antigen specificity can be exact at the nucleotide and protein level or mutated relative to the parent population by mutations at the nucleotide level (and by extension the protein level). This enables the body to have sufficient numbers of antigen-specific lymphocytes to mount an effective immune response.

The term “cytokines” refers to a wide variety of intercellular regulatory proteins produced by many different cells in the body which ultimately control every aspect of body defense. Cytokines activate and deactivate phagocytes and immune defense cells, increase or decrease the functions of the different immune defense cells, and promote or inhibit a variety of nonspecific body defenses.

The phrase “T4-helper lymphocytes”, also referred to as helper cells, refer to a type of white blood cell that orchestrate the immune response and enhance the activities of the killer T-cells (those that destroy pathogens) and B cells (antibody and immunoglobulin producers).

The phrase “affinity maturation” refers to the gradual modification of the paratope and entire B cell receptor as a result of somatic hypermutation. B lymphocytes with higher affinity B cell receptors that can 1) bind the epitope more tightly and 2) therefore bind the epitope for a longer period of time are able to proliferate more and survive longer. These B cells can eventually differentiate into plasma cells, which secrete their antibodies and form the basis of serum-mediated immunity.

The phrase “somatic hypermutation” (SHM) refers to a cellular mechanism by which the adaptive immune system adapts to foreign elements confronting it (e.g. viruses, bacteria, biomolecules). A major component of the process of affinity maturation, SHM diversifies B cell receptors used to recognize foreign elements (antigens) and allows the immune system to adapt its response to new threats during the lifetime of an organism. Somatic hypermutation involves a programmed process of mutation predominantly affecting select framework and complementarity-determining regions of immunoglobulin genes. Unlike germline mutation, SHM operates at the level of an organism's individual immune cells. These mutations are not transmitted to the organism's offspring, but are transmitted to daughter cells of individual B cell clones. Mistargeted somatic hypermutation is a likely mechanism in the development of B cell lymphomas and many other cancers. Somatic hypermutation can also lead to the acquisition of non-VDJ template DNA within B cell receptor sequences, such as LAIR1 insertions in malaria-specific neutralizing antibodies.

Somatic hypermutation is a distinct diversification mechanism from isotype switching (also called class switching). Mutations acquired during somatic hypermutation eventually lead to isotype switching, in which a B cell's antibody can be coupled to different functions by switching to a different Fc/constant region sequence. Isotype switching is an irreversible process, in that once a B cell has switched from a given constant region (e.g. IGHM) to a new constant region (e.g. IGHA1) it can no longer use the IgM constant region as the DNA encoding the IgM Fc is excised and removed during isotype switching.

The term “contig”, originating from the term “contiguous”, refers to a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequences and to overlapping physical segments (fragments) contained in clones depending on the context. Note that clone, in reference to overlapping clones, refers to individual bacteria or constructs (e.g. phagemids, cosmids, etc.) containing distinct insertions of genomes that were utilized in early efforts to map genomes.

The phrase “heavy chain” refers to the large polypeptide subunit of an antibody (immunoglobulin). The first recombination event to occur is between one D and one J gene segment of the heavy chain locus. Any chromosomal DNA between these two gene segments is deleted. This D-J recombination is followed by the joining of one V gene segment, from a region upstream of the newly formed DJ complex, forming a rearranged VDJ gene segment. All other gene segments between V and D segments are now deleted from the cell's genome. Primary transcript (unspliced RNA) is generated containing the VDJ region of the heavy chain and both the constant mu and delta chains (Cμ and Cδ) (i.e., the primary transcript contains the segments: V-D-J-Cμ-Cδ). The primary RNA is processed to add a polyadenylated (poly-A) tail after the Cμ chain and to remove sequence between the VDJ segment and this constant gene segment. Translation of this mRNA leads to the production of the IgM heavy chain protein and the IgD heavy chain protein (its splice variant). Expression of the immunoglobulin heavy chain with one or more surrogate light chains constitutes the pre-B cell receptor that allows a B cell to undergo selection and maturation.

The phrase “light chain” refers to the small polypeptide subunit of an antibody (immunoglobulin). The kappa (η) and lambda (λ) chains of the immunoglobulin light chain loci rearrange in a very similar way, except that the light chains lack a D segment. In other words, the first step of recombination for the light chains involves the joining of the V and J chains to give a VJ complex before the addition of the constant chain gene during primary transcription. Translation of the spliced mRNA for either the kappa or lambda chains results in formation of the Ig η or Ig λ light chain protein. Assembly of the Ig μ heavy chain and one of the light chains results in the formation of membrane bound form of the immunoglobulin IgM that is expressed on the surface of the immature B cell. B cells may express up to two heavy chains and/or two light chains in respectively rare and uncommon instances through a phenomenon known as allelic inclusion. This phenomenon can only be directly observed using single-cell technologies, though it can be inferred with a degree of uncertainty using a combination of bulk sequencing technologies and probabilistic inference via an extension of the birthday paradox.

The phrase “complementarity-determining regions” (CDRs) refers to part of the variable chains in immunoglobulins (antibodies) and T cell receptors, generated by B cells and T cells respectively, where these molecules are particularly hypervariable. The antigen-binding site of most antibodies and T cell receptors is typically distributed across these CDRs, collectively forming a paratope. However, there are many documented examples of paratopes that enable antigen recognition that fall outside of the CDRs. As the most variable parts of the molecules, CDRs are crucial to the diversity of antigen specificities and immune cell receptor sequences generated by lymphocytes.

In some aspects, the methods and systems described herein can provide for the determination of the sequence of long individual nucleic acid molecules and/or the identification of direct molecular linkage as between two sequence segments separated by long stretches of sequence, which permit the identification and use of long range sequence information, wherein such sequencing information is obtained using methods that have the advantages of the extremely low sequencing error rates and high throughput of short read sequencing technologies. The methods and systems described herein can segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long range molecular context can be preserved through a sequencing process, and can be preserved through the targeted enrichment process used in targeted sequencing approaches described herein, where no other sequencing approach has shown this ability.

In some aspects, sequence information from smaller fragments may retain the original long range molecular sequence context through the use of a tagging procedure, including the addition of barcodes as described herein or known in the art. In specific examples, fragments originating from the same original longer individual nucleic acid molecule can be tagged with a common barcode, such that any later sequence reads from those fragments can be attributed to that originating longer individual nucleic acid molecule. Such barcodes can be added using any method known in the art, including addition of barcode sequences during amplification methods that amplify segments of the individual nucleic acid molecules as well as insertion of barcodes into the original individual nucleic acid molecules using transposons, including methods such as those described in Amini et al., Nature Genetics 46: 1343-1349 (2014) (advance online publication on Oct. 29, 2014), which is hereby incorporated by reference in its entirety for all purposes and in particular for all teachings related to adding adaptor and other oligonucleotides using transposons. Once nucleic acids have been tagged using such methods, the resultant tagged fragments can be enriched using methods described herein such that the population of fragments represents targeted regions of the genome. As such, sequence reads from that population allows for targeted sequencing of select regions of the genome, and those sequence reads can also be attributed to the originating nucleic acid molecules, thus preserving the original long range molecular sequence context. The sequence reads can be obtained using any sequencing methods and platforms known in the art and described herein. In some aspects, such methods and systems are useful for assembly of complete VDJ sequences.

Methods of processing and sequencing nucleic acids in accordance with the methods and systems described in the present application are also described in further detail in U.S. Ser. Nos. 14/316,383; WO2015200893, WO2018119447 and WO2018075693 which are herein incorporated by reference in their entirety for all purposes and in particular for all written description, figures and working examples directed to processing nucleic acids and sequencing and other characterizations of genomic material.

In general, the methods and systems described herein accomplish sequencing of nucleic acid molecules including, but not limited to, DNA (e.g., genomic DNA), RNA (e.g., mRNA, including full-length mRNA transcripts, and small RNAs, such as miRNA, tRNA, and rRNA), and cDNA. In various embodiments, the methods and systems described herein accomplish genomic sequencing of nucleic acid molecules (e.g., DNA, RNA, and mRNA). In various embodiments, the methods and systems described herein accomplish genomic sequencing of immune cell receptor sequences (e.g., DNA, RNA, and mRNA). In various embodiments, the methods and systems described herein can accomplish transcriptome sequencing, e.g., whole transcriptome sequencing of mRNA encoding immune cell receptors. In some embodiments, the methods and systems described herein can also accomplish targeted genomic sequencing of nucleic acid molecules (e.g., DNA, RNA, and mRNA). In various embodiments, the methods and systems described herein accomplish single cell genomic sequencing, for example, single cell genomic sequencing of nucleic acid molecules (e.g., RNA and mRNA) encoding immune cell receptors of single cells, such as B cell receptors (BCRs) and T cell receptors (TCRs).

In various embodiments, the methods and systems described herein can include high-throughput sequencing technologies, e.g., high-throughput DNA and RNA sequencing technologies. In various embodiments, the methods and systems described herein can include high-throughput, higher accuracy short-read DNA and RNA sequencing technologies. In various embodiments, the methods and systems described herein can include long-read RNA sequencing, e.g., by sequencing cDNA transcripts in their entirety without assembly. In various embodiments, the methods and systems described herein can also, for example, segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long-range molecular context is not only preserved through a sequencing process, but is also preserved through the targeted enrichment process used in targeted sequencing approaches.

In general, the methods and systems described herein are directed to single cell analysis (including single- and multi-modal analyses) of genomic sequencing of nucleic acids (e.g., RNA and mRNA) encoding immune cell receptors of single cells, such as B cell receptors (BCRs) and T cell receptors (TCRs). Single cell analysis, including single cell multi-modal analyses (e.g., single cell immune cell receptor sequencing combined with, for example, gene expression, protein expression, and/or antigen capture technologies), as well as processing and sequencing of nucleic acids, in accordance with the methods and systems described in the present application are described in further detail, for example, in U.S. Pat. Nos. 9,689,024; 9,701,998; 10,011,872; 10,221,442; 10,337,061; 10,550,429; 10,273,541; and U.S. Pat. Pub. 20180105808, which are all herein incorporated by reference in their entirety for all purposes and in particular for all written description, figures and working examples directed to processing nucleic acids and sequencing and other characterizations of genomic material.

V(D)J recombination is a genetic recombination mechanism that occurs in developing lymphocytes during the early stages of T and B cell maturation. Through somatic recombination, this mechanism produces a highly diverse repertoire of antibodies/immunoglobulins and T cell receptors (TCRs) found in B cells and T cells, respectively. This process is a defining feature of the adaptive immune system and these receptors are defining features of adaptive immune cells.

V(DD)J recombination is a genetic recombination mechanism that, while discovered decades earlier, was not truly understood until recently given its non-adherence to classical rules to V(D)J recombination. However, understanding this mechanism have been a clear need, since tandem fusions of D-D genes can result in long CDR3s (24+ amino acids) or ultralong CDR3s (28+ amino acids). Though relatively rare, these long CDR3s are very biologically relevant, as they can be found in broadly neutralizing antibodies. See, for reference, https://www.ncbi.nlm.nih.gov/pmc/articles/PMC7605257/.

V(D)J recombination occurs in the primary lymphoid organs (bone marrow for B cells and thymus for T cells) and in a generally random fashion. The process leads to the rearranging of variable (V), joining (J), and in some cases, diversity (D) gene segments. As discussed above, the heavy chain possesses numerous V, D, and J gene segments, while the light chain possesses only V and J gene segments. The process ultimately results in novel amino acid sequences in the antigen-binding regions of immunoglobulins and TCRs that allow for the recognition of antigens from nearly all pathogens including, for example, bacteria, viruses, and parasites. Furthermore, the recognition can also be allergic in nature or may match host tissues and lead to autoimmunity.

Human antibody molecules, including B cell receptors (BCRs), include both heavy and light chains, each of which contains both constant (C) and variable (V) regions, and are genetically encoded on three loci. The first is the immunoglobulin heavy locus on chromosome 14, containing the gene segments for the immunoglobulin heavy chain. The second is the immunoglobulin kappa (η) locus on chromosome 2, containing the gene segments for part of the immunoglobulin light chain. The third is the immunoglobulin lambda (λ) locus on chromosome 22, containing the gene segments for the remainder of the immunoglobulin light chain.

Each heavy or light chain contains multiple copies of different types of gene segments for the variable regions of the antibody proteins. For example, the human immunoglobulin heavy chain region contains two C gene segments (Cμ and Cδ), 44 V gene segments, 27 D gene segments and 6 J gene segments. The number of given segments present in any individual can vary, as these gene segments are carried in haplotypes; for this reason, inference of both the alleles present within an individuals and the germline sequence of those alleles is an important step in correctly identifying B cell clonotypes. The light chains possess two C gene segments (Cλ and Cη) and numerous V and J gene segments, but do not have D gene segments. DNA rearrangement causes one copy of each type of gene segment to mate with any given lymphocyte, generating a substantial antibody repertoire. Approximately 10¹⁴ combinations are possible, with 1.5×10² to 3×10³ potentially removed via self-reactivity.

Accordingly, each naïve B cell makes an antibody with a unique Fab site through a series of gene recombinations, and later mutations, with the specific molecules of the given antibody attaching to the B cell's surface as a B cell receptor (BCR). These BCRs are then available to react with epitopes of an antigen.

When the immune system encounters an antigen, epitopes of that antigen will be presented to many B lymphocytes. B lymphocytes first rearrange a heavy chain that enables pre-B cell receptor ligand binding. B lymphocytes that bind multivalent self-targets after rearrangement of the light chain too strongly are eliminated and die or undergo a secondary recombination event, while B cells that do not bind self-targets too strongly are licensed to exit the bone marrow. The latter becomes available to respond to non-self antigens and to undergo clonal expansion. This process is known as clonal selection.

Cytokines produced by activated T4-helper lymphocytes enable those activated B-lymphocytes (B cells) to rapidly proliferate to produce large clones of thousands of identical B cells. More specifically, when under threat (i.e., via bacteria, virus, etc.), the body releases white blood cells by the immune system. The T4 lymphocytes help the response to a threat by triggering the maturation of other types of white blood cell. They produce special proteins, called cytokines, have plural functions, including the ability to summon all of the other immune cells to the area, and also the ability to cause nearby cells to differentiate (become specialized) into mature B cells and T-cells.

Accordingly, while only a few B cells in the body may have an antibody molecule that can bind a particular epitope, eventually many thousands of cells are produced with the right specificity, allowing the body's immune system to act en masse. This is referred to as clonal expansion. Natural phenomena such as IgA deficiency and murine transgenic models have shown that there are multiple paths by which a B cell receptor can acquire novel antigen specificity even from a very limited repertoire through the processes of somatic hypermutation and affinity maturation.

As the B cells proliferate, they undergo affinity maturation as a result of somatic hypermutation. This allows the B cells to “fine-tune” the paratopes of the antibody to more effectively fit with the recognized epitopes. B cells with high affinity B cell receptors on their surface bind epitopes more tightly and for a longer period of time, which enables these cells to selectively proliferate. Over the course of this proliferation and expansion, these variant B cells differentiate into plasma cells that synthesize and secrete vast quantities of antibodies with Fab sites that fit the target epitopes very precisely.

The phrase “immune cell” refers to a cell that is part of the immune system and that helps the body fight infections and other diseases. Immune cells include innate immune cells (such as basophils, dendritic cells, neutrophils, etc.) that are the first line of body's defense and are deployed to help attack the invading foreign cells (e.g., cancer cells) and pathogens. The innate immune cells can quickly respond to foreign cells and pathogens to fight infection, battle a virus, or defend the body against bacteria. Immune cells can also include adaptive immune cells (such as lymphocytes including B cells and T cells). The adaptive immune cells can come into action when an invading foreign cells or pathogens slip through the first line of body's defense mechanism. The adaptive immune cells can take longer to develop, because their behaviors evolve from learned experiences, but they can tend to live longer than innate immune cells. Adaptive immune cells remember foreign invaders after their first encounter and fight them off the next time they enter the body. Both types of immune cells employ important natural defenses in helping the body fight foreign cells and pathogens for fighting infections and other diseases.

Accordingly, the immune cells of the disclosure can include, but are not limited to, neutrophils, eosinophils, basophils, mast cells, monocytes, macrophages, dendritic cells, natural killer cells, and lymphocytes (such as B cells and T cells). The immune cells of the disclosure can further include dual expresser cells or DE (such as unique dual-receptor-expressing lymphocytes that co-express functional B cell receptor (BCR) and T cell receptor (TCR)), cells with adaptive immune receptors that may diversify or may not diversify (including immune cells expressing a chimeric antigen receptor with a fixed nucleotide sequence or with the capacity to mutate), and TCR co-expressors (i.e., hybrid αβ-γδ T cells) that co-express both αβ and γδ TCR chains.

The phrase “immune cell receptor”, “immune receptor”, or “immunologic receptor” refers to a receptor or immune cell receptor sequence, usually on a cell membrane, which can recognize components of pathogenic microorganisms (e.g., components of bacterial cell wall, bacterial flagella or viral nucleic acids) and foreign cells (e.g., cancer cells), which are foreign and not found naturally on the host cells, or binds to a target molecule (for example, a cytokine), and causes a response in the immune system. The immune cell receptors of the immune system can include, but are not limited to, pattern recognition receptors (PRRs), Toll-like receptors (TLRs), killer activated and killer inhibitor receptors (KARs and KIRs), complement receptors, Fc receptors, B cell receptors, and T cell receptors.

The phrase “immune cell receptor sequences” of an immune cell receptor include both heavy and light chains, each of which contains both constant (C) and variable (V) regions. For example, B cell receptors (BCRs) or B cell receptor sequences (including human antibody molecules) comprise of immunoglobulin heavy and light chains, each of which contains both constant (C) and variable (V) regions. Each heavy or light chain not only contains multiple copies of different types of gene segments for the variable regions of the antibody proteins, but also contains constant regions. For example, the BCR or human immunoglobulin heavy chain contains two (2) constant (Constant mu (Cμ) and delta (Cδ)) gene segments and forty four (44) Variable (V) gene segments, plus twenty seven (27) Diversity (D) gene segments, and six (6) Joining (J) gene segments. The BCR light chains also possess two (2) constant gene segments ((Constant lambda (Cλ) and kappa (Cη) and numerous V and J gene segments, but do not have any D gene segments. DNA rearrangement (i.e., recombination events) in developing B cells can cause one copy of each type of gene segment to go in any given lymphocyte, generating an enormous antibody repertoire. Accordingly, the primary transcript (unspliced RNA) of a BCR heavy chain can be generated containing the VDJ region of the heavy chain and both the constant mu and delta chains (Cμ and Cδ), i.e., the heavy chain primary transcript can contains the segments: V-D-J-Cμ-Cδ). In case of the B cell receptor and human immunoglobulin light chain, the first step of recombination for the light chains involves the joining of the V and J chains to give a VJ complex before the addition of the constant chain gene during primary transcription. Translation of the spliced mRNA for either the constant η (Cη) or λ (Cλ) chains results in formation of the Ig η or Igλ light chain protein.

In general, most T cell receptors (TCR) are composed of an alpha (α) chain and a beta (β) chain, each of which contains both constant (C) and variable (V) regions. Thus, the most common type of a T cell receptor is called an alpha-beta TCR because it is composed of two different chains, one α-chain and one beta β-chain. A less common type of TCR is the gamma-delta TCR, which contains a different set of chains, one gamma (γ) chain and one delta (δ) chain. The T cell receptor genes are similar to immunoglobulin genes for the BCR and undergo similar DNA rearrangement (i.e., recombination events) in developing T cells as for the B cells. For example, the alpha-beta TCR genes also contain multiple V, D, and J gene segments in their beta chains and V and J gene segments in their alpha chains, which are re-arranged during the development of the T cells to provide a cell with a unique T cell antigen receptor. Thus, the β-chain of the TCR can contain Vβ-Dβ-Jβ gene segments and constant domain (Cβ) genes resulting in a Vβ-Dβ-Jβ-Cβ sequence of the TCR β-chain. The re-arrangement of the alpha (α) chain of the TCR follows β chain rearrangement, and can include Vα-Jα gene segments and constant domain (Cα) genes resulting in a Vα-J α-Cα sequence of the TCR α-chain. Similar to the alpha-beta TCRs, the TCR-γ chain is produced by V-J recombinations and can contain Vγ-Jγ gene segments and constant domain (Cγ) genes resulting in a Vγ-Jγ-Cγ sequence of the TCR γ-chain, while the TCR-δ chain is produced using V-D-J recombinations, and can contain Vδ-Dδ-Jδ gene segments and constant domain (Cδ) genes resulting in a Vδ-Dδ-Jδ-Cδ sequence of the TCR δ-chain.

The phrase “immune cell receptor constant region sequence” or “immune receptor constant region sequence” refers to the constant region or constant region sequence of an immune cell receptor. For example, the immune cell receptor constant region sequence or immune receptor constant region sequence can include, but is not limited to, the constant mu (Cμ and delta (Cδ) region genes and sequences of a BCR and immunoglobulin heavy chain, the constant lambda (Cλ) and kappa (Cη) region genes and sequences of a BCR and immunoglobulin light chain, the alpha constant (Cα) region genes and sequences of a TCR α-chain sequence, the beta constant (Cβ) region genes and sequences of a TCR β-chain sequence, the gamma constant (Cγ) region genes and sequences of a TCR γ-chain sequence, and the delta constant (Cδ) region genes and sequences of a TCR δ-chain sequence.

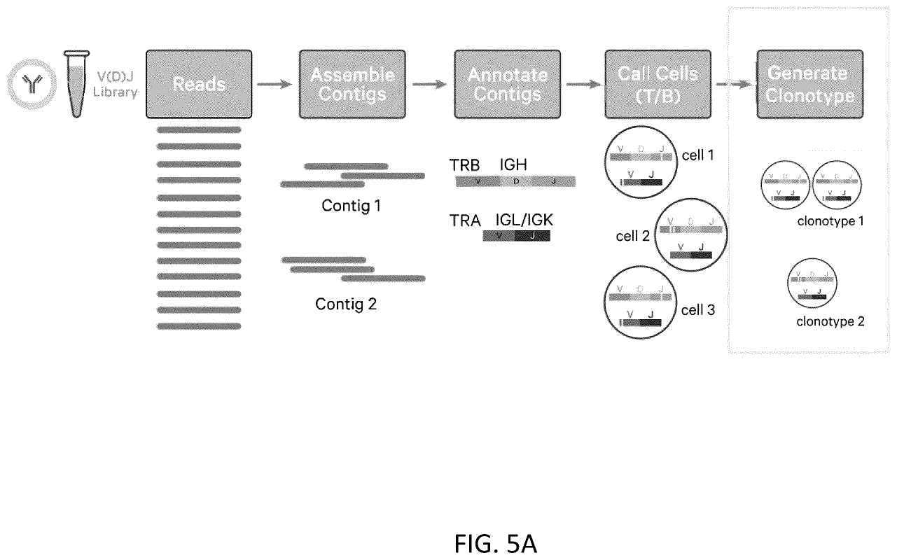

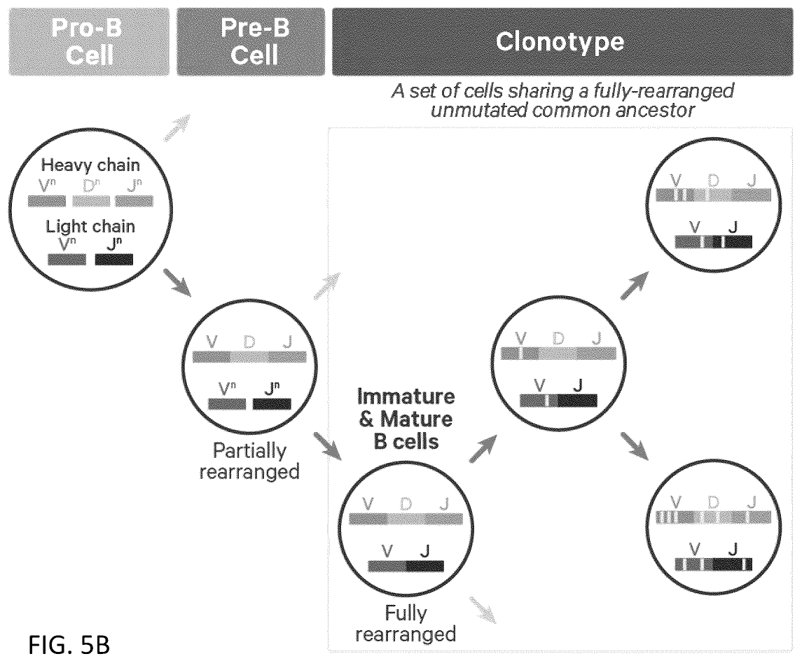

The general process of clonotyping is illustrated in FIG. 5A. In various embodiments, single cell analysis is performed to obtain a VDJ sequence library. In various embodiments, the sequence library is sequenced to obtain a plurality of reads. In various embodiments, the plurality of reads are aligned to a reference sequence.

In various embodiments, contigs are assembled. As used herein, a contig is a contiguous sequence of bases produced by assembly.

In various embodiments, contigs are annotated (e.g., with V, D, and/or J, and TRB, TRA, IGH, and/or IGL). In various embodiments, cells are called.

In various embodiments, during the clonotype grouping stage, cell barcodes are placed in groups called clonotypes. In various embodiments, each clonotype consists of all descendants of a single, fully rearranged common ancestor, as approximated computationally. In various embodiments, during this process, some cell barcodes are flagged as likely artifacts and filtered out, meaning that they are no longer called as cells. In various embodiments, nucleic acid sequence data (including one or more nucleic acid sequences) are provided as input to a VDJ alignment model. In various embodiments, the nucleic acid sequence data are provided in a FASTQ format. In various embodiments, the nucleic acid sequence data includes a barcode sequence, a name of the contig sequence, a nucleotide sequence of the contig, a contig quality score, a fraction of reads for this barcode that were provided as input to the assembly algorithm, a number of reads assigned to this contig, a number of UMIs assigned to this contig, a starting nucleotide base position of the start codon on the contig, a last nucleotide base position of stop codon on the contig, an amino acid sequence of the contig, an amino acid sequence of the contig's CDR3, a nucleotide sequence of the contig's CDR3, a starting base of the contig's CDR3, a last base of the contig's CDR3, start and stop positions of the contig's FWR1-FWR4 regions, start and stop positions of the contig's CDR1-CDR2 regions, annotations for the contig from the reference file, clonotype information, a TRUE or FALSE statement of whether the contig has high confidence, a list of UMIs that have been validated, a list of UMIs that have not been validated, a list of invalidated UMIs, a TRUE or FALSE statement about whether the barcode was declared a cell, a TRUE or FALSE statement about whether the contig was productive based on five criteria. NULL=not full length, a TRUE or FALSE statement about whether the barcode was declared a cell by gene expression data, a TRUE or FALSE statement about whether the barcode was declared a cell by the VDJ assembler, and/or a TRUE or FALSE statement about whether the contig is full length.

Germline sequences: In various embodiments, for each dataset, the reference sequence for V genes in the donor's genome (germline sequence) is derived to use as a reference for SHMs. In this context, a “donor” is an individual from whom adaptive immune cells (T cells, B cells) are collected (e.g. a sister and a brother would each be considered unique donors for the purposes of V(D)J aggregation).

In various embodiments, for each V segment, one cell from each approximated clonotype is chosen. In various embodiments, approximated clonotypes are not final clonotypes (i.e., those generated as the final step of the clonotype grouping algorithm). In various embodiments, the distribution of bases in each position on the V segment (excluding the last 15 bases) is determined. In various embodiments, a V gene position is considered a germline variant if a non-reference base is seen in at least 4 approximated clonotypes, comprising at least 25% of the total number of approximated clonotypes. In various embodiments, this process is repeated for all cells in all the approximated clonotypes. In various embodiments, the resulting cell-specific “footprint” defines alternative alleles. In various embodiments, there is no restriction on the number of possible alternative alleles. In various embodiments, germline variant assessment for J genes is currently not performed as it does not greatly enhance clonotype specificity.

Exact subclonotype grouping: In various embodiments, cells are placed into groupings called exact subclonotypes if they have identical VDJ transcripts. In this context, an exact subclonotype is a subset of cells within a clonotype that share identical immune receptor sequences at the nucleotide level, spanning the entirety of the V, D, and J genes and the V(D)J junction. Exact subclonotypes share the same V, D, J, and C gene annotations (e.g. cells that have identical V(D)J sequences but different C genes or isotypes are split into distinct exact subclonotypes).

In various embodiments, only productive contigs are used. A contig is termed productive if the following conditions are met: 1) Full length requirement—the contig matches the initial part of a V gene, and the contig continues on, ultimately matching the terminal part of a J gene; 2) Start requirement—the initial part of the V matches a start codon on the contig (in the human and mouse reference sequences as described herein, every V segment begins with a start codon); 3) Nonstop requirement—there is no stop codon between the V start and the J stop; 4) In-frame requirement—the J stop minus the V start equals one mod three, meaning that the codons on the V and J segments are in frame; 5) CDR3 requirement—there is an annotated CDR3 sequence (as described below); 6) Structure requirement—let VJ denote the sum of the lengths of the V and J segments, let len denote the J stop minus the V start, measured on the contig, then VJ—len lies between −25 and +25, except for IGH, which are between −55 and +25. This condition is imposed to preclude anomalous structure changes that are unlikely to correspond to functional proteins.

For each contig, a CDR3 sequence is searched for using the conserved sequence that flanks the CDR3 region. Then the CDR3 sequence and its flanking regions are compared to motifs derived from V and J reference segments for human and mouse, as shown below. A letter represents a specific amino acid and a dot represents any amino acid.

| | | |
|---|---|---|
| left flank | CDR3 | right flank |
| LQPEDSAVYY | C . . . | LTFG.GTRVTV |
| VEASQTGTYF | | LIWG.GSKLSI |
| ATSGQASLYL | | |

In this embodiment, a CDR3 sequence has at least 5 amino acids, starts with a C, and does not contain a stop codon. The flanking sequences for a candidate CDR3 are matched against the above motifs, and scored+1 for each position that matches one of the entries in a column. For example, LTY . . . scores 2 for the first three amino acids in the right flank. L matches an entry in the first column, contributing 1 to the score. T matches an entry in the second column, contributing 1 to the score. Y does not match the third column, and does not contribute to the score. In this embodiment, for a candidate CDR3 to be declared a CDR3 sequence, it scores at least 10. In addition, the left flank contributes at least 3 and the right flank contributes at least 4.

Next, the implied stop position of the end of the V segment is found on the contig. The implied stop is the start position of the V segment on the contig plus the length of the V segment. The CDR3 sequence starts at most 10 bases before the stop, and at most 20 bases after the stop of the V. These conditions for finding an implied stop are not applied in the denovo case.

If there is more than one CDR3 sequence, the one with the highest score is chosen. If there is a tie, the one with the later start position on the contig is chosen. If a tie remains, the longer CDR3 is chosen.

In various embodiments, exact subclonotypes have the same number of chains. In various embodiments, exact subclonotypes must also be identical in their VDJ sequences and constant region gene assignments. In various embodiments, exact subclonotypes are not required to have identical 5′ UTRs. In various embodiments, the algorithm does not test for SHM in the 5′ UTR or constant region.

Joining exact subclonotypes into clonotypes: In various embodiments, exact subclonotypes are iteratively merged into clonotypes based on comparing each pair of exact subclonotypes to each other. In various embodiments, two cells with set criteria of shared differences and minimal CDR3 mutations are deemed to be in the same clonotype. In various embodiments, merging criteria are briefly described here. In various embodiments, pairs of exact subclonotypes having 2-3 chains are considered for joining together into a clonotype. In various embodiments, later stages of the clonotype grouping algorithm evaluate and merge exact subclonotypes with 1 chain. In various embodiments, exact subclonotypes having 4 chains (putative doublets) are not joined. In various embodiments, two exact subclonotypes are merged if a pair of chains has V-J genes and CDR3 segments of identical length. In various embodiments, shared somatic hypermutations (SHM) in V-J sequence outside the junction regions are identified between different exact subclonotypes. In various embodiments, a mutation is shared if the two chains carry the same substitution or indel with respect to the reference sequence (donor reference for V and universal reference for J). In various embodiments, using the donor reference sequences enables the exclusion of shared germline mutations. In various embodiments, chains that have too many CDR3 mutations are discarded based on a set threshold. For example, in some embodiments, a constant N is used, with cd1 being set to the number of heavy chain CDR3 nucleotide differences, and cd2 set to the number of light chain CDR3 nucleotide differences. Let n1 be the nucleotide length of the heavy chain CDR3, and likewise n2 for the light chain. Then N=80\hat{ }(42*(cd1/n1+cd2/n2)). The number 80 may be alternately specified via MULT_POW and the number 42 via CDR3_NORMAL_LEN. CDR3 nucleotide identity of at least 85% is required for exact subclonotype retention.

Clonotype and barcode filtering: In various embodiments, during library generation, artifacts can arise by two mechanisms. In the first mechanism, reverse transcription or sequencing can introduce base call errors. These usually occur at bases having low quality scores. In various embodiments, cells with these low-quality bases are screened out, typically at a low rate. In the second mechanism, Gel Beads-in-emulsion (GEMs) may contain material from two or more cells: entire intact cells, cell fragments, or individual mRNA molecules. In various embodiments, contamination detection is a complex task and is accomplished via multiple heuristic filters. In various embodiments, some barcode filtering happens during the assembly and cell calling stages. In various embodiments, filtering and clonotype grouping happen simultaneously.

In various embodiments, default filters are applied. In various embodiments, one or more filter are recursive. Example of filters include: a cell filter that remove barcodes not called cells in the pipeline; a maximum contigs filter that remove barcodes with more than four productive contigs; a graph filter that remove some exact subclonotypes that appear to be background; a cross filter that uses cross-library information (i.e., from two libraries originating from the same donor) To remove spurious exact subclonotypes; a barcode duplication filter that removes duplicated barcodes within an exact subclonotype; a whitelist filter that identifies and removes any artifactual barcodes that do not match a barcode in a barcode whitelist (artifactual barcodes are rare and likely arise from Gel Bead contamination); a foursie filter that removes some four-chain clonotypes that are biologically irrelevant, e.g., 4 heavy chains; an improper filter that removes exact subclonotypes having 3 or 4 identical chains; a weak onesie filter that disintegrates some single-chain clonotypes into single cells (if a barcode has a high confidence contig, passes the cell calling filter, and has only 1 chain, it is retained as its own clonotype); a UMI filter that determines a baseline UMI count for each dataset and remove any B cells having UMI counts lower than this baseline (helps eliminate rare clonotype expansion signatures arising from fragmentation of plasma cells or other poorly understood physical processes); a UMI ratio filter that remove some B cells with low UMI counts, relative to mean UMI counts in a given clonotype; a GEX filter that removes barcodes that were called as cells in the VDJ but not the GEX library (this filter mitigates any overcalling issues seen in BCR and TCR libraries); a doublet filter that remove some barcodes that appear to represent doublets or higher-order multiplets; a signature filter that removes some exact subclonotypes that appear to represent contaminants, based on their chain signature (as some complex clonotypes with many chains represent multiple true clonotypes that are glued together into a single clonotype); a onesie merger that prevents the merger of some single-chain clonotypes into other clonotypes; a weak chain filter that, from the remaining cells, remove any cells that have weak chains (a chain is weak if it is found in ≤5 other cells, and the total number of cells in that clonotype is less than 5 times that number, e.g., if there are a total of 14 cells in a clonotype, and a given chain is found in only 3 of those cells, all 3 cells are filtered out. However, if there were at least 3×5 (15 cells) in the clonotype, the 3 cells with this chain would be retained); and/or a quality merger that filters out exact subclonotypes with low quality score positions.

Initial grouping: In various embodiments, for each pair of exact subclonotypes, and for each pair of chains in each of the two exact subclonotypes, for which V . . . J has the same length for the corresponding chains, and the CDR3 segments have the same length for the corresponding chains, the exact subclonotypes are considered for joining into the same clonotype.

Shared mutations: enclone next finds shared mutations between exact subclonotypes, that is, for two exact subclonotypes, common mutations from the reference sequence, using the donor reference for the V segments and the universal reference for the J segments. Shared mutations are supposed to be somatic hypermutations, that would be evidence of common ancestry. By using the donor reference sequences, most shared germline mutations are excluded, and this is critical for the algorithm's success.

Are there enough shared mutations? We find the probability p that “the shared mutations occur by chance”. More specifically, given d shared mutations, and k total mutations (across the two cells), we compute the probability p that a sample with replacement of k items from a set whose size is the total number of bases in the V . . . J segments, yields at most k-d distinct elements. The probability is an approximation, stirling number of the second kind.

Too many CDR3 mutations: In various embodiments, a constant N is defined where N=80\hat{ }(42*(cd1/n1+cd2/n2)). In various embodiments, cd1 is set to the number of heavy chain CDR3 nucleotide differences, cd2 is set to the number of light chain CDR3 nucleotide differences, n1 is the nucleotide length of the heavy chain CDR3, and n2 is the nucleotide length of the light chain. In various embodiments, the CDR3 nucleotide identity is required to be at least a predetermined threshold. For example, the predetermined threshold may be 80%, 85%, 90%, 92.5%, 95%, etc. In various embodiments, the nucleotide identity is determined by dividing cd by the total nucleotide length of the heavy and light chains, normalized.

Key join criteria: In various embodiments, two cells sharing sufficiently many shared differences and sufficiently few CDR3 differences are deemed to be in the same clonotype. That is, the lower p is, and the lower N is, the more likely it is that the shared mutations represent bona fide shared ancestry. In various embodiments, the smaller p*N is, the more likely it is that two cells lie in the same true clonotype. In various embodiments, to join two cells into the same clonotype, the bound p*n≤C is required to be satisfied, where C is a constant (e.g., 100,000). In various embodiments, this constant may be determined by empirically balancing sensitivity and specificity across a large collection of datasets.

Other join criteria: In various embodiments, if V gene names are different (after removing trailing * . . . ), and either V gene reference sequences are different, after truncation on right to the same length or 5′ UTR reference sequences are different, after truncation on left to the same length, then the join is rejected. In various embodiments, as an exception to the key join criterion, a join which has at least a predetermined number of shares (e.g., 15) is allowed, even if p*N>C. In various embodiments, as a second exception to the key join criterion, heavy chain join complexity may be determined by finding the optimal D gene, allowing no D, or DD), and aligning the junction region on the contig to the concatenated reference. In various embodiments, the heavy chain join complexity h_{comp} is then a sum as follows: each inserted base counts one, each substitution counts one, and each deletion (regardless of length) counts one. Then we allow a join if it has h_{comp}−cd≥8, so long as the number of differences between both chains outside the junction regions is at most 80, even if p*N>C. In various embodiments, two clonotypes which were assigned different reference sequences are not joined unless those reference sequences differ by at most a predetermined number of positions (e.g., 2 positions). In various embodiments, there is an additional restriction imposed when creating two-cell clonotypes, that cd≤d, where cd is the number of CDR3 differences and d is the number of shared mutations, as above. In various embodiments, this filter may be turned off. In various embodiments, cases where light chain constant regions are different and cd>0 are not joined. In various embodiments, a join is rejected if the percent nucleotide identity on heavy chain FWR1 is at least 20 more than the percent nucleotide identity on heavy chain CDR1+CDR2 (combined). In various embodiments, in cases where there is too high a concentration of changes in the junction region, no join is performed. More specifically, if the number of mutations in CDR3 is at least 5 times the number of non-shared mutations outside CDR3 (maxed with 1), the join is rejected.

In various embodiments, two exact subclonotypes can be joined if they have the same V and J gene assignments, the same CDR3 lengths, and CDR3 nucleotide identity of at least a predetermined threshold (e.g., 75%, 80%, 85%, 90%, 92.5%, 95%, etc.) on each chain.

In various embodiments, the lack of somatic hypermutation (SHM) in T cell receptors (TCRs) yields biological clonotypes that have identical V(D)J transcripts. In various embodiments, fully rearranged B cell receptors (BCRs) can undergo SHM, which can increase antigen affinity. Thus for BCRs, VDJ transcripts in a clonotype can differ at any position, as shown in FIG. 5B. In various embodiments, B cell clonotypes can be hard to infer accurately because SHM can introduce numerous mutations. In various embodiments, B cell clonotype grouping is performed by simultaneously filtering and grouping cells into clonotypes, as described in more detail below.

To understand what constitutes members of a clonotype, one can start with the original progenitor cell for a given lineage of B cells, this progenitor cell commonly referred to as the parent clone, which is a single cell to which all daughter cells will be genetically related, though their B cell receptors and exact antigen specificity may differ. Collectively, this parent clone and all its daughter cells constitute a clonotype. As stated above, accurate identification of the members of a clonotype is critical not just from a biological perspective, but also from the biomedical perspective, as correct identification of all of the members of a given clonotype can be useful in the identification and discovery of therapeutic antibodies, design of vaccines (e.g., what antibody lineages can be expanded by a vaccine or are expanded successfully or unsuccessfully by a vaccine), in the monitoring of B cell-mediated immune disease (e.g., myasthenia gravis and lupus), and in other settings. Known approaches that attempt to group immune cell receptor sequences into groups with shared antigen specificity or members of the same clonotype include 1) immcantation, 2) Clonify, 3) GLIPH, 4) TCRdist, 5) VDJTools, 6) MiXCR, 7) AbSolve, 8) PMID 23536288, PMID 23898164, PMID 25345460. While some of these algorithms can successfully identify groups of T cells with shared antigen specificity using single-cell data (TCRdist, GLIPH), and the other algorithms use solely bulk receptor sequencing data (i.e., without access to heavy and light chain sequences), none of these algorithms attempt to approximate the true clonotype for B cells while also attempting to mitigate for sources of noise in the data.

With this understanding of the immune cell's purpose in fighting off attacking foreign antigens, the pharmaceutical industry has strongly focused on developing antibody therapeutics or designing vaccines with the ability to expand antibody lineages directed towards specific B cells with shared antigen specificity. To most effectively determine the efficacy of a vaccine or antibody therapeutic, it is essential to be able to accurately identify cell members of a clonotype, which potentially share common or similar BCRs or antigen specificity. The pharmaceutical industry has also directed its efforts to isolate antibodies and antibody lineages against non-foreign targets for the purpose of developing antibody-based therapeutics for a broad array of disease states including autoimmune disease (anti-inflammatory targets), cancer (checkpoint inhibitors and other targets), and other conditions such as osteoporosis. Similarly, knowing the fine specificities of different antibody lineages elicited by a vaccine is key to understanding serum neutralization profiles and global epitope maps of an entire virus. This same concept applies to understanding how a patient's adaptive immune system can render drugs such as adalimumab ineffective through the emergence of anti-drug antibodies and distinct anti-drug antibody lineage.

Therefore, in accordance with various embodiments, various methods and systems are provided for identifying a D gene segment in a VDJ sequence.

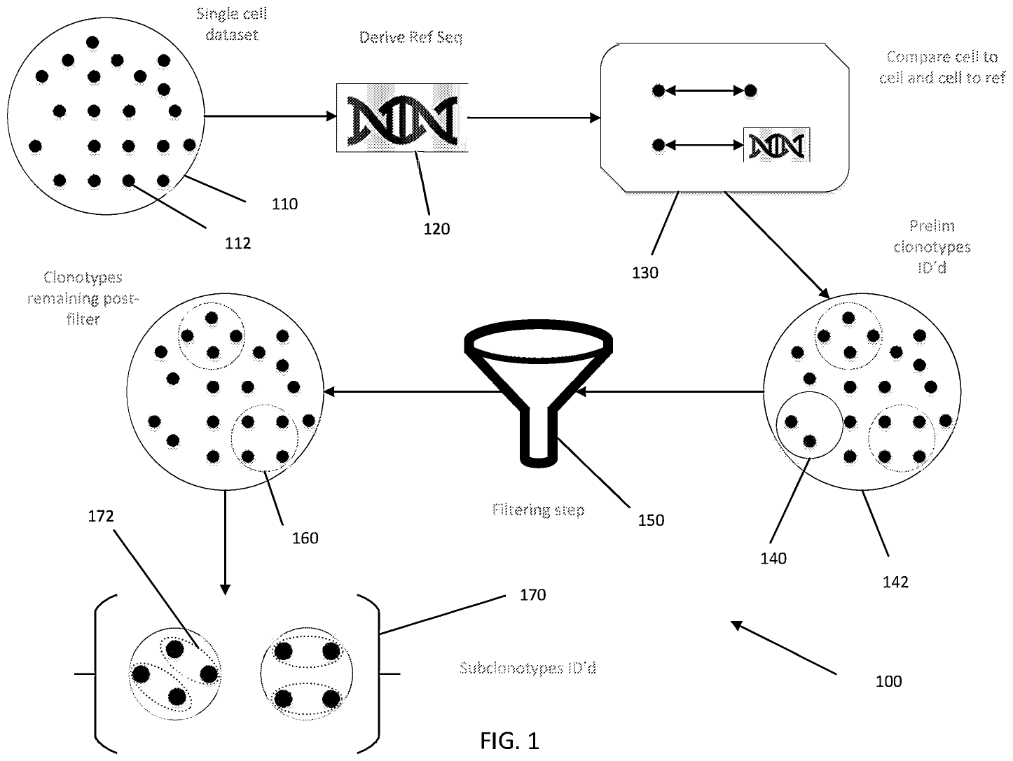

FIG. 1—General Workflow

In accordance with various embodiments, a general schematic workflow is provided in FIG. 1 to illustrate a non-limiting example process for grouping lymphoid cells within a lymphoid cell variable domain region sequence dataset. The workflow can include various combinations of features, whether it be more or less features than that illustrated in FIG. 1. As such, FIG. 1 simply illustrates one example of a possible workflow.

FIG. 1 provides a schematic workflow 100, the workflow including an immune receptor 110. In some embodiments, the immune receptor dataset 110 can comprise VDJ sequence information. In some embodiments, the immune receptor dataset 110 can be a variable domain region sequence data set, e.g., obtained from a cell sample comprising VDJ expressing cells, e.g., a cell sample comprising a plurality of lymphoid cells 112. More detail regarding the acquisition of said dataset 110 will be provided below. From that dataset, a reference variable domain region sequence 120 is identified. Reference variable domain region sequence 120 can be a donor reference sequence, universal reference sequence, or both. More detail regarding the acquisition of the reference variable domain region sequence 120, as well as further discussion related to the donor reference sequence and universal reference sequence will be provided below.

With dataset 110 and reference sequence(s) 120 in hand, one or more comparisons 130 may be conducted. These comparisons can include comparing the variable domain region sequences associated with the lymphoid cells of the dataset. Various cell to cell comparisons can be contemplated here and will be discussed in further detail below. These comparisons can also include comparing the variable domain region sequences of the various lymphoid cells to the reference variable domain region sequence. Again, various reference to cell comparisons can be contemplated here and will be discussed in further detail below. It should be understood, and will be discussed below, that both comparisons are individually beneficial for grouping purposes, but can also be done together as part of the workflow.

Based on the one or more comparisons 130, one or more clonotypes 140 can be identified from dataset 110, as part of an identification protocol 142. Via identification protocol 142, the identification of clonotypes 140 is subject to meeting one or more comparison criteria. Detail regarding how comparisons 130, via the one or more comparison criteria, can lead to identification of the one or more clonotypes 140, will be provided below.

Identified clonotypes 140 can also be subject to one or more filters 150 that can function to remove specific cells from identified clonotypes, or eliminate whole clonotypes, that do not meet specific comparison criteria or are filtered out via the constraints imposed by the one or more filters 150. Detail regarding the filters will be provided below. Again, it should be understood that FIG. 1 simply illustrates a non-limiting example of the process for grouping lymphoid cells. As such, the one or more filters 150 can activate after clonotypes are identified. Alternatively, the one or more filters can activate as part of identification protocol 142. Moreover, it is contemplated that one or more of filters 150 can activate before identification protocol 142. Even further, there need not be any active filters as part of the workflow 100.

Regardless of when or if one or more filters 150 are activated, an updated set of clonotypes 160 can be identified. As illustrated in FIG. 1, after application of filter(s) 150, two clonotypes 160 remained of the three originally identified clonotypes 140. It is understood, however, that in accordance with various embodiments, the one of more filters 150 need not be used, and that identification of the updated set of clonotypes 160 need not occur.

Regardless of when or if one or more filters 150 are activated, identified clonotype members can then be subcategorized into subclonotypes 172 as part of a subclonotype identification protocol 170. Per the above, the one or more clonotypes 140 identified from dataset 110 as part of an identification protocol 142 can proceed directly to a subclonotype identification protocol 170. Alternatively, as illustrated in FIG. 1, clonotypes 160 remaining after activation of filters 150 can proceed to the subclonotype identification protocol 170. With identification of clonotypes and subclonotypes in hand, these results can then be output, as desired, for user review.

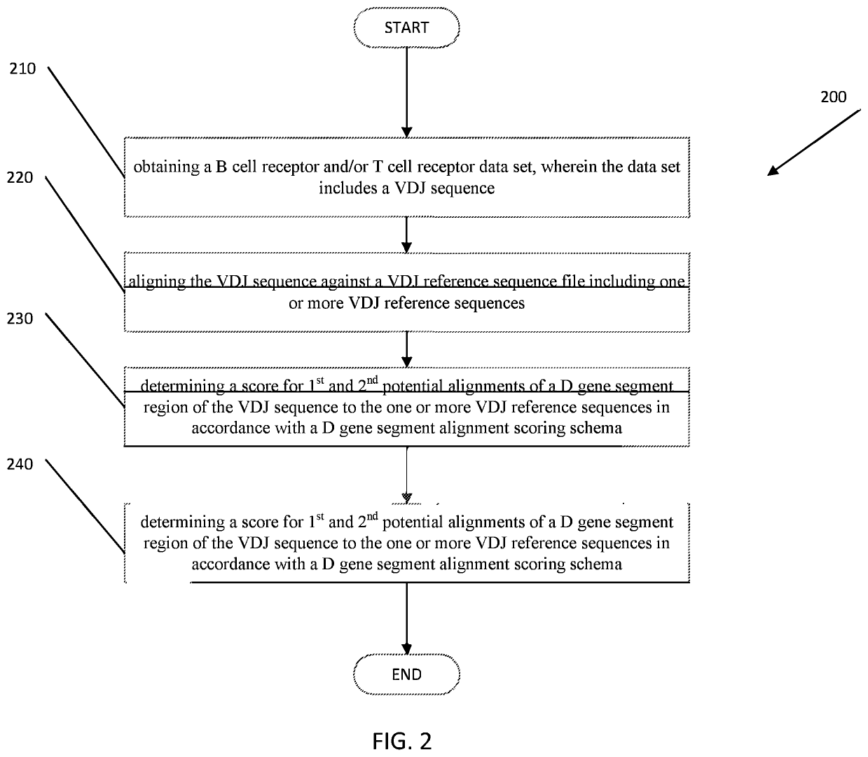

In accordance with various embodiments, an example method 200 for identifying a D gene segment in a VDJ sequence is illustrated in FIG. 2. Method 200 can include a step 210, which includes obtaining a B cell receptor and/or T cell receptor data set, wherein the data set includes a VDJ sequence.

Method 200 can further include a step 220, which includes aligning the VDJ sequence against a VDJ reference sequence file including one or more VDJ reference sequences.

Method 200 can further include a step 230, which includes determining a score or 1^{st} and 2^{nd} potential alignments of a D gene segment region of the VDJ sequence to the one or more VDJ reference sequences in accordance with a D gene segment alignment scoring schema.

Method 200 can further include a step 240, which includes identifying the potential alignment with a score exceeding a pre-determined threshold as a potential correct alignment of the D gene segment region of the VDJ sequence.

While method 200 of FIG. 2 illustrates one example method for identifying a D gene segment in a VDJ sequence, it should be noted that various methods for identifying a D gene segment in a VDJ sequence are contemplated herein, and can include various combinations of steps discussed herein. This applies to non-transitory computer-readable medium, as well, in which a program is stored for causing a computer to perform a method for identifying a D gene segment in a VDJ sequence, as discussed herein. This further applies to systems for identifying a D gene segment in a VDJ sequence, as discussed herein.

In accordance with various embodiments, the various methods for identifying a D gene segment in a VDJ sequence can further include applying a pre-determined scoring adjustment factor to the score of the 1^{st} and 2^{nd} potential alignments of the D gene segment region for the VDJ sequence.

In accordance with various embodiments, the various methods for identifying a D gene segment in a VDJ sequence can further include identifying the potential alignment with the highest score as a correct alignment of the D gene segment region.

In accordance with various embodiments, the scoring schema can (or can be configured to) add points to the score for each base match of a potential alignment of the D gene segment region to the reference VDJ sequence. The scoring schema can (or can be configured to), in various embodiments, subtract points from the score for each base mismatch of a potential alignment of the D gene segment region to the reference VDJ sequence.

In various embodiments, a nucleic acid sequence (e.g., DNA) representing a VDJ sequence is compared to a reference nucleic acid sequence (e.g., a reference VDJ sequence). In various embodiments, the nucleic acid sequence is obtained from single cell analysis and represents a VDJ sequence from a single cell. In various embodiments, the comparison between the obtained nucleic acid sequence and the reference nucleic acid sequence is base-by-base. In various embodiments, a match score is generated by pairwise sequence alignment. In various embodiments, a scoring matrix is determined when comparing two sequences. In various embodiments, scoring parameters are used to generated scores in the matrix. In various embodiments, the scoring parameters include at least one of: match, mismatch, gap open for insertion between VDJ segments, gap open for deletion bridging VDJ segments, gap open (otherwise), gap extend for insertion between VDJ segments, and/or gap extend (otherwise). In various embodiments, the value for each parameter may be selected from the range of −20 to +20. In various embodiments, the value for each parameter may be selected from the range of −19 to +19. In various embodiments, the value for each parameter may be selected from the range of −18 to +18. In various embodiments, the value for each parameter may be selected from the range of −17 to +17. In various embodiments, the value for each parameter may be selected from the range of −16 to +16. In various embodiments, the value for each parameter may be selected from the range of −15 to +15. In various embodiments, the value for each parameter may be selected from the range of −14 to +14. In various embodiments, the value for each parameter may be selected from the range of −13 to +13. In various embodiments, the value for each parameter may be selected from the range of −12 to +12. In various embodiments, the value for each parameter may be selected from the range of −12 to +11. In various embodiments, the value for each parameter may be selected from the range of −12 to +10. In various embodiments, the value for each parameter may be selected from the range of −12 to +9. In various embodiments, the value for each parameter may be selected from the range of −12 to +8. In various embodiments, the value for each parameter may be selected from the range of −12 to +7. In various embodiments, the value for each parameter may be selected from the range of −12 to +6. In various embodiments, the value for each parameter may be selected from the range of −12 to +5. In various embodiments, the value for each parameter may be selected from the range of −12 to +4. In various embodiments, the value for each parameter may be selected from the range of −12 to +3. In various embodiments, the value for each parameter may be selected from the range of −12 to +2. For example, a match is scored at +2, a mismatch is scored at −2, a gap open for insertion between VDJ segments is scored at −4, a gap open for deletion bridging VDJ segments is scored at −4, a gap open (otherwise) is scored at −12, a gap extend for insertion between VDJ segments is scored at a −1, and a gap extend (otherwise) is scored at a −2.

The scoring schema can (or can be configured to), in various embodiments, subtract points from the score for each gap that has to be opened for insertions in between V and D sequences and D and J sequences of a potential alignment of the D gene segment region to the reference VDJ sequence. The scoring schema can (or can be configured to), in various embodiments, subtract points from the score for each gap extension in between the V and D sequences and D and J sequences of a potential alignment of the D gene segment region to the reference VDJ sequence. The scoring schema can (or can be configured to), in various embodiments, subtract points from the score for each gap that has to be deleted to close the gap between V and D sequences and D and J sequences of a potential alignment of the D gene segment region to the reference VDJ sequence.

In various embodiments, a gap scoring function is constant and assigns a constant penalty (e.g., −1) for each gap position. In various embodiments, the gap scoring function is a convex function and penalizes each additional position in the gap less than the previous position in the gap. In various embodiments, the gap scoring function is an affine gap penalty function that assigns a first penalty to open a gap and a second penalty to extending a gap. In various embodiments, the first penalty is greater than the second penalty. In various embodiments, the gap penalty function includes positional penalty rules. In various embodiments, the gap penalty function includes one or more penalties (e.g., gap open, gap extend, etc.) specific to different regions of a nucleic acid sequence (e.g., V region, D or DD region, J region, junctions between the VDJ regions, etc.). In the example above, the gap penalty function assigns a −4 for a gap open for insertion between VDJ segments or a gap open for deletion bridging VDJ segments while all other gap opens outside of the aforementioned regions are assigned a higher penalty of −12.

The scoring schema can (or can be configured to), in various embodiments, subtract points from the score for all gap openings outside of the V-D-J junction of a potential alignment of the D gene segment region to the reference VDJ sequence. The scoring schema can (or can be configured to), in various embodiments, subtract points from the score for all other gap extensions of a potential alignment of the D gene segment region to the reference VDJ sequence.

In accordance with various embodiments, a non-transitory computer-readable medium is provided, in which a program is stored for causing a computer to perform a method for identifying a D gene segment in a VDJ sequence. The method can include, for example, obtaining a B cell receptor and/or T cell receptor data set, wherein the data set includes a VDJ sequence. The method can also include, for example, aligning the VDJ sequence against a VDJ reference sequence file including one or more VDJ reference sequences. The method can further include, for example, determining a score for 1^{st} and 2^{nd} potential alignments of a D gene segment region of the VDJ sequence to the one or more VDJ reference sequences in accordance with a D gene segment alignment scoring schema. The method can also include, for example, identifying the potential alignment with a score exceeding a pre-determined threshold as a potential correct alignment of the D gene segment region of the VDJ sequence.

In accordance with various embodiments, the various non-transitory computer-readable media, in which a program is stored for causing a computer to perform a method for identifying a D gene segment in a VDJ sequence, can further include applying a pre-determined scoring adjustment factor to the score of the 1^{st} and 2^{nd} potential alignments of the D gene segment region for the VDJ sequence.

In accordance with various embodiments, the various non-transitory computer-readable media, in which a program is stored for causing a computer to perform a method for identifying a D gene segment in a VDJ sequence, can further include identifying the potential alignment with the highest score as a correct alignment of the D gene segment region.

In accordance with various embodiments, the scoring schema can (or can be configured to) add points to the score for each base match of a potential alignment of the D gene segment region to the reference VDJ sequence. The scoring schema can (or can be configured to), in various embodiments, subtract points from the score for each base mismatch of a potential alignment of the D gene segment region to the reference VDJ sequence.

The scoring schema can (or can be configured to), in various embodiments, subtract points from the score for each gap that has to be opened for insertions in between V and D sequences and D and J sequences of a potential alignment of the D gene segment region to the reference VDJ sequence. The scoring schema can (or can be configured to), in various embodiments, subtract points from the score for each gap extension in between the V and D sequences and D and J sequences of a potential alignment of the D gene segment region to the reference VDJ sequence. The scoring schema can (or can be configured to), in various embodiments, subtract points from the score for each gap that has to be deleted to close the gap between V and D sequences and D and J sequences of a potential alignment of the D gene segment region to the reference VDJ sequence.

The scoring schema can (or can be configured to), in various embodiments, subtract points from the score for all gap openings outside of the V-D-J junction of a potential alignment of the D gene segment region to the reference VDJ sequence. The scoring schema can (or can be configured to), in various embodiments, subtract points from the score for all other gap extensions of a potential alignment of the D gene segment region to the reference VDJ sequence.

In summary, and in accordance with various embodiments herein, the systems and methods exemplified herein solve multiple problems, including how to (a) pick the “best” reference D segment, for example in the case of immunoglobulin heavy chain (IGH) or T Cell Receptor Beta Locus (TRB), and (b) exhibit the “correct” alignment of the transcript to the concatenated reference. The various methods herein provide for the alignment of the V(D)J region on a transcript to the concatenated V(D)J reference, allowing for each possible D reference segment (or the null D segment, or DD), such as for IGH or TRB. In various embodiments, D genes can be assigned to each IGH or TRB exact subclonotype. Every such exact subclonotype can be assigned the optimal D gene, or two D genes configuration (in a VDDJ clonotype), or none, depending on score. (The none case is applied only when no insertion is observed.) The algorithm aligns the V(D)J region on a transcript to the concatenated V(D)J reference, allowing for each possible D reference segment (or the null D segment, or DD in a V(DD)J clonotype), in the case of IGH or TRB.

These alignments can be carried out using the following example of one non-limiting scoring scheme, reflected in Table 1 below.

TABLE 1

| Case | Match |
|---|---|
| Match | 2 |
| Mismatch | −2 |
| gap open for insertion between V/D/J segments | −4 |
| gap open for deletion bridging V/D/J segments | −4 |
| gap open (otherwise) | −12 |
| gap extend for insertion between V/D/J segments | −1 |
| gap extend (otherwise) | −2 |

To determine the score from the provided Table 1, for example, an alignment is generated using an alignment algorithm (global or local), e.g., pairwise alignment algorithm (e.g., Smith-Waterman, Needleman-Wunsch, word methods (i.e., k-tuple methods), maximal unique match, Hirschberg's, Hamming Distance, Landau-Vishkin, Myers' bit vector, etc.). In various embodiments, 2.2 times a bit score is added (measures sequence similarity independent of query sequence length and database size and is normalized based on the raw pairwise alignment score) for the alignment. In various embodiments, the bit score is defined as −log2 of the probability that a random DNA sequence of length n will match a given DNA sequence with ≤k mismatches=sum{1=0 . . . =k} (n choose 1)*3\hat{ }1/4\hat{ }n. The alignment and its score are both then edited. In various embodiments, the D segment having the highest score is selected. In various embodiments, a D segment is arbitrarily (e.g., randomly) selected in the case of a tie.

The following parameters can be optimized in designing the algorithm: the inconsistency rate for a large dataset (over a million cells), placement of indels (manual examination), and consistency with IgBLAST, or if not, justifiable difference from it.

To assess the inconsistency rate, if one allows clonotypes having a large number of exact subclonotypes, then measurement can be noisy because a single clonotype can overly influence the rate. For this reason, one can restrict to clonotypes having at most 10 exact subclonotypes. Moreover, since recomputing with very large data sets is too time consuming, not only can one set a maximum exact clonotype ceiling, one can set a minimum exact clonotype floor to produce a set range of clonotype count, the output of which can be further processed by applying an inconsistency parameter to the output to identify the number (or percentage) of clonotypes having D-gene assignment inconsistencies.

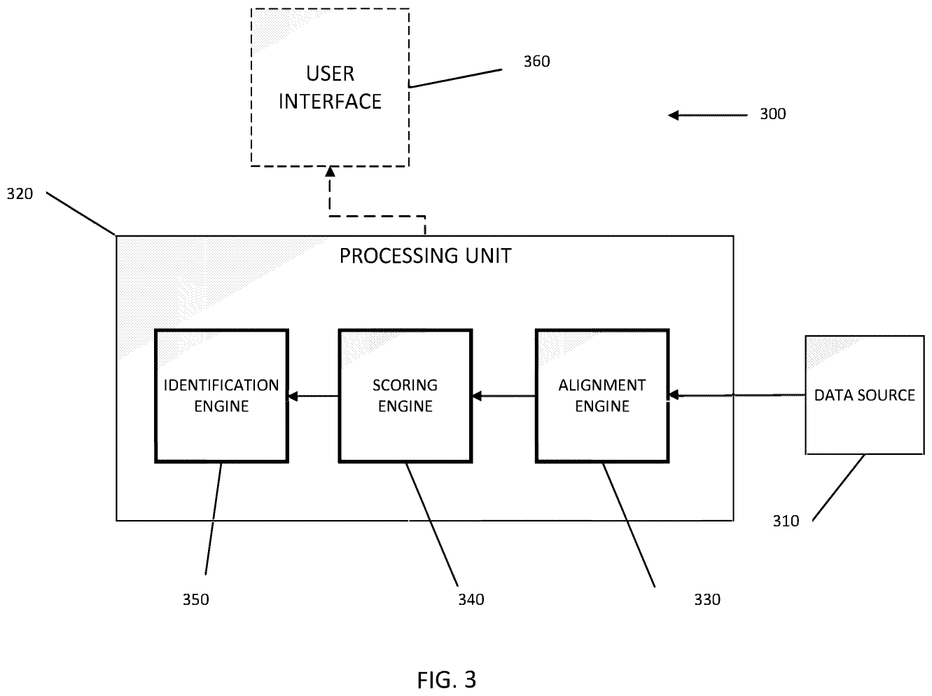

In accordance with various embodiments, an example system 300 for identifying a D gene segment in a VDJ sequence is illustrated in FIG. 3. System 300 can include a data source 310 and a processing unit 320. Processing unit 320 can include one or more of, for example, an alignment engine 330, a scoring engine 340, and an identification engine 350. System 300 can also include a user interface 360.

Note that all previous discussion of additional features, particularly with regard to the preceding described methods and non-transitory computer-readable media, in accordance with various embodiments, are applicable to the features of the various system embodiments described and contemplated herein.

Data source 310 can be configured to obtain a B cell receptor and/or T cell receptor data set, wherein the data set includes a VDJ sequence

Processing unit 320 can be configured to receive the B cell receptor and/or T cell receptor data set from the data source.

As stated above, processing unit 320 can further include one or more of, for example, alignment engine 330, scoring engine 340, and identification engine 350. In various embodiments, alignment engine 330 can be configured to align the VDJ sequence against a VDJ reference sequence file including one or more VDJ reference sequences.

In various embodiments, scoring engine 340 can be configured to determine a score for 1^{st} and 2^{nd} potential alignments of a D gene segment region of the VDJ sequence to the one or more VDJ reference sequences in accordance with a D gene segment alignment scoring schema. In various embodiments, scoring engine 340 can be configured to apply a pre-determined scoring adjustment factor to the score of the 1^{st} and 2^{nd} potential alignments of the D gene segment region for the VDJ sequence. In various embodiments, scoring engine can be configured to identify the potential alignment with the highest score as a correct alignment of the D gene segment region.

Identification engine 350 can be configured to identify the potential alignment with a score exceeding a pre-determined threshold as a potential correct alignment of the D gene segment region of the VDJ sequence.

In accordance with various embodiments, the scoring schema can (or can be configured to) add points to the score for each base match of a potential alignment of the D gene segment region to the reference VDJ sequence. The scoring schema can (or can be configured to), in various embodiments, subtract points from the score for each base mismatch of a potential alignment of the D gene segment region to the reference VDJ sequence.

The scoring schema can (or can be configured to), in various embodiments, subtract points from the score for each gap that has to be opened for insertions in between V and D sequences and D and J sequences of a potential alignment of the D gene segment region to the reference VDJ sequence. The scoring schema can (or can be configured to), in various embodiments, subtract points from the score for each gap extension in between the V and D sequences and D and J sequences of a potential alignment of the D gene segment region to the reference VDJ sequence. The scoring schema can (or can be configured to), in various embodiments, subtract points from the score for each gap that has to be deleted to close the gap between V and D sequences and D and J sequences of a potential alignment of the D gene segment region to the reference VDJ sequence.

The scoring schema can (or can be configured to), in various embodiments, subtract points from the score for all gap openings outside of the V-D-J junction of a potential alignment of the D gene segment region to the reference VDJ sequence. The scoring schema can (or can be configured to), in various embodiments, subtract points from the score for all other gap extensions of a potential alignment of the D gene segment region to the reference VDJ sequence.

In accordance with various embodiments, processing unit 320 of system 300 of FIG. 3 can be communicatively connected to data source 310 and user interface 360. In various embodiments, and as stated above, processing unit 320 can include alignment engine 330, scoring engine 340, and identification engine 350. It should be appreciated that each component (e.g., engine, module, unit, etc.) depicted as part of system 300 (and described herein) can be implemented as hardware, firmware, software, or any combination thereof.

In various embodiments, processing unit 320 can be implemented as an integrated instrument system assembly with data source 310, or user interface 360, or both. That is, any combination of processing unit 320, data source 310 and user interface 360 can be housed in the same housing assembly and communicate via conventional device/component connection means (e.g., serial bus, optical cabling, electrical cabling, etc.).

In various embodiments, processing unit 320 can be implemented as a standalone computing device (as shown in FIG. 3) that can be communicatively connected to the data source 310 (and likewise user interface 360) via an optical, serial port, network or modem connection. For example, the processing unit 320 can be connected via a LAN or WAN connection that allows for the transmission of data to and from the data source 310, and likewise user interface 360.

In various embodiments, the functions of processing unit 320 can be implemented on a distributed network of shared computer processing resources (such as a cloud computing network) that is communicatively connected to the data source 310 via a WAN (or equivalent) connection. For example, the functionalities of processing unit 320 can be divided up to be implemented in one or more computing nodes on a cloud processing service such as AMAZON WEB SERVICES™.

Within the processing unit 320, alignment engine 330, scoring engine 340, and identification engine 350 can be implemented as separate engines, as illustrated in FIG. 3 and described in the example provided above. However, it should readily be understood that the features and configurations described above in relation to alignment engine 330, scoring engine 340, and identification engine 350, can be interchanged in any combination between the engines or wholly housed in one or the other engines. It should also be recognized that alignment engine 330, scoring engine 340, and identification engine 350 and be implemented as a single engine, possessing all the capabilities discussed herein in relation to alignment engine 330, scoring engine 340, and identification engine 350 individually. As such, FIG. 3 simply provides one example implementation of a system in accordance with various embodiments, and should be not be read to limit the interchangeability, interoperability and/or functionality of all the components therein.

### Data Acquisition

In accordance with various embodiments, systems and methods within the disclosure include obtaining a dataset. That dataset can be a sequence dataset. The sequence dataset can be a lymphoid cell sequence dataset. The lymphoid cell sequence dataset can be a B cell receptor and/or a T cell receptor data set. The lymphoid cell sequence dataset can be a variable domain region sequence dataset. The dataset can include plurality of variable domain region sequences including both heavy chain region and light chain region sequences of antibodies and immunoglobulins, T-cell receptors (TCRs), or B-cell receptors (BCRs). The sequences in the dataset can represent the heavy chain variable region and light chain variable region sequences for each individual lymphoid cell in a sample. The lymphoid cell can be a B cell or a T cell.

The B cell and T cell variable domain regions of the heavy and light chains contain multiple copies of V, J, and in some instances D gene segments for the variable regions of the antibody proteins. The variable domain region of the heavy chain contains V, D, and J gene segments, whereas the variable domain region of the light chain contains only V and J gene segments and lack a D gene segment. Accordingly, the lymphoid cell variable domain region sequence dataset includes light chain sequences containing the V and J segments and heavy chain sequences containing the V, D and J segments.

The sample can be any biological sample, including for example, blood, tissue, cells, cell cultures, urine, or saliva. Another example of a sample can be a tube of cells from a donor or subject, from a particular tissue at a particular point in time, and possibly enriched for particular cells. The terms donor and subject are used interchangeably herein. A donor or a subject is an individual from which samples are obtained. The donor or subject can be a mammalian subject, including for example, a human, swine, monkey, ape, dog, cat, mouse (e.g., a humanized mouse), or rat. In some embodiments, the sample can be a splenocyte sample, a lymphocyte sample, or a bone marrow sample obtained from a mammalian subject.

As discussed herein, various sequencing technologies can be used to obtain the dataset sequences from the cells in a sample. The sequencing technologies can include next generation sequencing (NGS) technology. One example of the next generation sequencing technology can be 10× Genomics' Chromium™ single-cell RNA-sequencing technology. The Chromium™ single-cell RNA-sequencing technology takes samples containing cells of interests (e.g., a lymphoid cell such as a B cell or a T cell), and uses microfluidic partitioning to capture single cells in the sample and prepares uniquely barcoded, beads called Gel Bead-In Emulsions (GEMs), which are then used to derive barcoded cDNA libraries and sequenced by Illumina® sequencing instruments to generate the sequencing output data. As discussed herein, the various embodiments of Chromium™ single-cell RNA-sequencing technology within the disclosure can at least include platforms such as One Sample, One GEM Well, One Flowcell; One Sample, One GEM well, Multiple Flowcells; One Sample, Multiple GEM Wells, One Flowcell; and Multiple Samples, Multiple GEM Wells, One Flowcell platform. Accordingly, as discussed herein, the various embodiments within the disclosure can include, for example, a sequence dataset from one or more biological samples, biological samples from one or more donors, and multiple libraries from one or more donors. It is understood that other sequencing technologies and platforms are also contemplated within the disclosure for generating the sequence output data from lymphoid cell samples.

The various embodiments, systems and methods within the disclosure further include processing and inputting the sequence output data, for example, the Chromium™ single-cell RNA-sequence output data discussed above. As an example, a compatible format of the sequencing data can be as FASTQ files. One example of a software tool that processes and inputs the sequencing output data for producing the dataset within the disclosure can be the Cell Ranger™ Software. The Cell Ranger™ Software processes the Chromium single-cell RNA-sequence output data and transforms the sequencing output data into input dataset ready for analysis by the various embodiments, systems and methods within the disclosure. Accordingly, as an example within the disclosure, a dataset can include all sequencing data obtained from a particular library type (e.g., TCR or BCR), from one cell group, processed by running thorough the Cell Ranger™ Software pipeline. It is understood that other software tools are also contemplated within the disclosure for processing and transforming the sequencing output data into input files.

### Reference Sequence Determination

In accordance with various embodiments, systems and methods within the disclosure can further include identifying a reference sequence (e.g., VDJ reference sequence file) such as, for example, a variable domain region sequence. The reference variable domain region sequence can be a donor reference sequence, universal reference sequence, or both. It should be noted that the quality of universal reference sequences can vary drastically across species and depends on human annotations (which can be quite variable in quality) and the underlying genome assemblies (which can also be quite variable in quality).

In accordance with various embodiments, the donor reference sequence can be derived for each of the heavy and light chain V segments by genotyping or estimating the genotype of the V segments from the dataset. The donor reference sequence, when derived for each of the heavy and light chain V segments by genotyping the V segments from the dataset, represent the V chains present in the donor's genome. The information related to the V segments is presumed to be imperfect because V segments vary in their expression frequency, and therefore, large number of cells are required for the information to be complete. In other words, the more cells are present, the more complete the information will be with respect to the donor reference sequence for the heavy and light chain V segments. The second reason that the information related to the V segments is presumed to be imperfect is because it is not always possible to accurately determine the last ˜15 bases in a V chain from transcript data.

In accordance with various embodiments within the disclosure, the universal reference sequence can include a sequence found in a public database. The universal sequence can often be the single sequence for a given genomic segment that is found in the reference sequence for the given species. Accordingly, it can be presumed that a donor reference sequence is a modified version of the universal reference sequence that has mutations introduced, that are believed to have arisen in the germline sequence of the donor. As an example within the disclosure, the universal reference sequence can include J segment portions of the variable domain region sequences, D segment portions of the variable domain region sequences, or both.

### Cell Comparison and Grouping

In accordance with various embodiments, one or more comparison criteria can be utilized to sufficiently identify clonotypes or subclonotypes. These criteria need not be utilized as a group. It is understood that, certain criterion can be used independently or in combination with other steps discussed herein, while other criterion can only be used in combination with other steps discussed herein, in accordance with various embodiments within the disclosure. It is also understood that the criteria discussed below are simply examples, and not exhaustive. As such, the possible comparison criteria should not be limited to just those discussed herein. Comparison criteria can include, for example, computing the germline alleles for the donor's V segments, separately joining singletons, two libraries from the same tube of cells, same length V and J portion and predetermined threshold of nucleotide differences, same length CDRs and maximum number of nucleotide differences, same barcode with at least two cells, and comparison with the reference and shared mutations. For two cell clonotypes, comparison criteria can also include, for example, determining the number of CDR differences between the cell members, and determining that the comparison criteria is not met if the number of CDR differences exceed a determined two-cell threshold. Two-cell clonotype can be determined at least by cd≤d/2 (where cd is the number of differences between the given CDR3 nucleotide sequences and d are shared mutations between the two cells). In accordance with various embodiments, the two-cell threshold therefore can have a value dependent on the number of shared mutations.

Further, in accordance with various embodiments, various system and methods can further include identifying subclonotypes within an identified clonotype. The subclonotype includes cells having identical V(D)J transcripts. The subclonotype can further include cells having an identical C segment, same distance between a J stop codon and a C start codon, or both. The subclonotype can include cells having two or three chains.

### Noise Filtering

In accordance with various embodiments, systems and methods can further include filters that, when activated, can provide the user more refined output.

An example of a filter is a cross-filter. If one specifies that two or more libraries arose from the same sample (i.e., from the same tube of cells), then the default behavior of the various embodiments herein, can be to “cross filter” so as to remove expanded exact subclonotypes that are present in one library but not another, in a fashion that would be highly improbable, assuming random draws of cells from the tube. Such observed behavior can be understood to arise when a plasma or plasmablast cell breaks up during or after pipetting from the tube, and the resulting fragments seed can yielding ‘fake’ cells. This filter, presumably defaulted to being on during sample analysis of subclonotype identification, can also be turned off per user input. It is understood that the reverse is also contemplated.

Another example of a filter relates to a filter that, by default in various embodiments, removes exact subclonotypes that by virtue of their relationship to other exact subclonotypes, appear to arise from background mRNA or a phenotypically similar phenomenon. This filter, presumably defaulted to being on during sample analysis of subclonotype identification, can also be turned off per user input. It is understood that the reverse is also contemplated.

Another example of a filter relates to a filter that, by default in various embodiments, filters out exact subclonotypes having a base in VJ that looks like it might be wrong. A Phred quality score (Q score) is a measure of the quality of the identification of the nucleobases generated by automated DNA sequencing. Various methods, in accordance with various embodiments herein, can find bases which are not Q60 for a barcode, not Q40 for two barcodes, are not supported by other exact subclonotypes, are variant within the clonotype, and which disagree with the donor reference. This filter, presumably defaulted to being on during sample analysis of subclonotype identification, can also be turned off per user input. It is understood that the reverse is also contemplated.

Another example of a filter relates to a filter that, by default in various embodiments, filters out chains from clonotypes that are weak and appear to be artifacts, perhaps arising from, for example, a stray mRNA molecule. This filter, presumably defaulted to being on during sample analysis of subclonotype identification, can also be turned off per user input. It is understood that the reverse is also contemplated.

Another example of a filter relates to a filter that, by default in various embodiments, filters out onesie clonotypes (a clonotype or exact subclonotype having exactly one chain) having a single exact subclonotype, and that are light chain or TRA gene, and whose number of cells is less than, for example, 0.1% of the total number of cells. This filter, presumably defaulted to being on during sample analysis of subclonotype identification, can also be turned off per user input. It is understood that the reverse is also contemplated.

Another example of a filter relates to a filter that, by default in various embodiments, finds a foursie exact subclonotype that contains a twosie exact subclonotype having at least ten cells, it kills the foursie exact subclonotype, no matter how many cells it has. The foursies that are killed are believed to be rare odd artifacts arising from repeated cell doublets or, for example, GEMs (gel bead in emulsion) that contain two cells and multiple gel beads. This filter, presumably defaulted to being on during sample analysis of subclonotype identification, can also be turned off per user input. It is understood that the reverse is also contemplated.

Another example of a filter relates to a filter that, by default in various embodiments, filters out rare artifacts arising from contamination of oligos on gel beads. This filter, presumably defaulted to being on during sample analysis of subclonotype identification, can also be turned off per user input. It is understood that the reverse is also contemplated.

Another example of a filter relates to a filter that, by default in various embodiments, labels an exact subclonotype as improper if it does not have one chain of each type. This filtering option causes all improper exact subclonotypes to be retained, although they may be removed by other filters.

Yet another example of a filter relates to a filter that, by default in various embodiments, deletes any exact subclonotype having less than n chains. Such a filter can be used to “purify” a clonotype so as to display only exact subclonotypes having all their chains. Similarly, another example of a filtering option relates to a filter that, by default in various embodiments, deletes any exact subclonotype having less than n cells. Such a filter can be used for a very large and complex expanded clonotype, for which it may be desired to see a simplified view.

Computer System

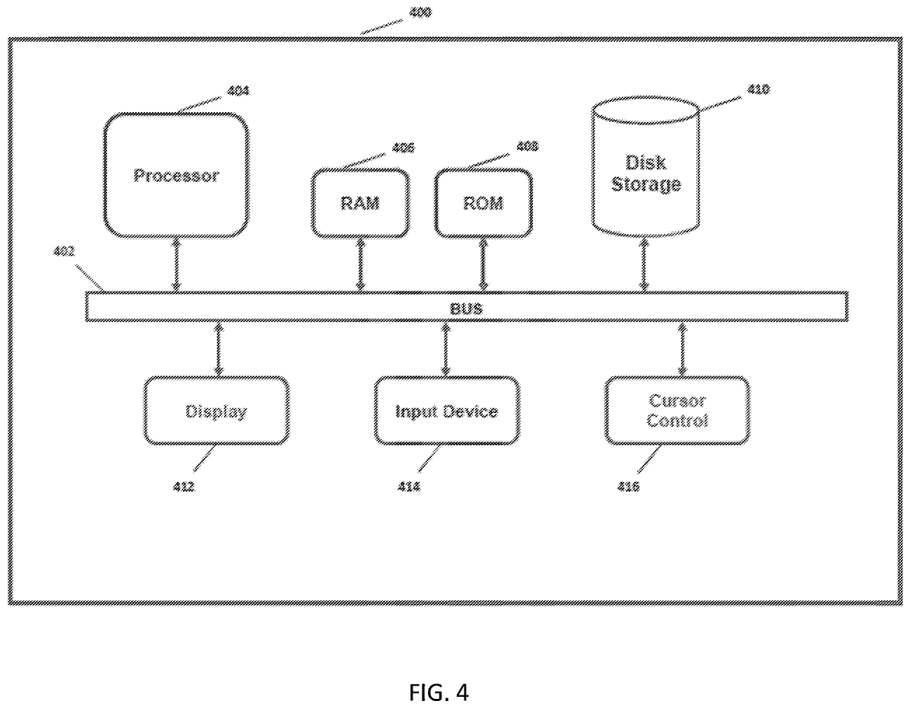

FIG. 4 is a block diagram that illustrates a computer system 400, upon which embodiments of the present teachings may be implemented. In various embodiments of the present teachings, computer system 400 can include a bus 402 or other communication mechanism for communicating information, and a processor 404 coupled with bus 402 for processing information. In various embodiments, computer system 400 can also include a memory, which can be a random access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a trackball or cursor direction keys for communicating direction information and command selections to processor 404 and for controlling cursor movement on display 412. This input device 414 typically has two degrees of freedom in two axes, a first axis (i.e., x) and a second axis (i.e., y), that allows the device to specify positions in a plane. However, it should be understood that input devices 414 allowing for 3 dimensional (x, y and z) cursor movement are also contemplated herein.

Consistent with certain implementations of the present teachings, results can be provided by computer system 400 in response to processor 404 executing one or more sequences of one or more instructions contained in memory 406. Such instructions can be read into memory 406 from another computer-readable medium or computer-readable storage medium, such as storage device 410. Execution of the sequences of instructions contained in memory 406 can cause processor 404 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as memory 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400 of Appendix D, whereby processor 404 would execute the analyses and determinations provided by these engines, subject to instructions provided by any one of, or a combination of, memory components 406/408/410 and user input provided via input device 414.

Digital Processing Device

In various embodiments, the systems and methods described herein can include a digital processing device, or use of the same. In various embodiments, the digital processing device can includes one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net- BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In some embodiments, the non-volatile memory comprises dynamic random-access memory (DRAM). In various embodiments, the non-volatile memory comprises ferroelectric random access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

Computer Program

In various embodiments, the systems and methods disclosed herein can include at least one computer program, or use at least one computer program. A computer program includes a sequence of instructions, executable in the digital processing device's CPU, written to perform a specified task. Computer readable instructions may be implemented as program modules, such as functions, objects, Application Programming Interfaces (APis), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® .NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or eXtensible Markup Language (XML). In various embodiments, a web application is written to some extent in a presentation definition language such as Cascading Style Sheets (CS S). In various embodiments, a web application is written to some extent in a client-side scripting language such as Asynchronous Javascript and XML (AJAX), Flash® Actionscript, Javascript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™ and Unity®.

Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelera-tor®, Celsius, Bedrock, Flash Lite, .NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nin-tendo DSi Shop.

Standalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often per-formed, at least in part, to create an executable program. In various embodiments, a computer program includes one or more executable complied applications.

Web Browser Plug-in

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object oriented databases, object databases, entity-relation-ship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

1. A method for identifying one or more D gene segment in a VDJ or VDDJ sequence, the method comprising:
obtaining a B cell receptor and/or T cell receptor data set, wherein the data set comprises a VDJ sequence;
aligning the VDJ sequence to one or more VDJ reference sequences thereby generating a first potential alignment and a second potential alignment;
determining a first score for the first potential alignment and a second score for the second potential alignment in accordance with a D gene segment alignment scoring schema; and
identifying a D gene segment region associated with a highest score between the first score and the second score.

2. The method of claim 1, wherein aligning the VDJ sequence to one or more VDJ reference sequences comprises applying a first affine gap penalty function when aligning regions between VDJ segments of the VDJ sequence and a second affine gap penalty function when aligning other regions of the VDJ sequence; and/or
wherein aligning comprises determining a first alignment score and a second alignment score.

3. The method of claim 2, wherein the first affine gap penalty function penalizes gap opens for insertion between VDJ segments at a first rate, and wherein the second affine gap penalty function penalizes gap opens for deletion bridging VDJ segments at a second rate, or penalizes other gap opens at a third rate that is larger than the first rate and the second rate, penalizes gap extends for insertion between VDJ segments at a fourth rate, and penalizes other gap extends at a fifth rate that is higher than the fourth rate.

4. The method of claim 1, further comprising:
applying a pre-determined scoring adjustment factor to the score of the 1^{st} and 2^{nd} potential alignments of the D gene segment region for the VDJ sequence; and/or
identifying the potential alignment with the highest score as a correct alignment of the D gene segment region; and/or
identifying an additional D gene segment, which is present in a VDDJ sequence.

5. (canceled)

6. (canceled)

7. The method of claim 1, wherein determining the first score comprises adding 2.2 times a first bit score to the first alignment score, wherein:
$$bit score = {\sum }_{l = 0}^{k} ( \begin{matrix}n \\ l\end{matrix} ) * \frac{{3}^{l}}{{4}^{n}}$$
where n is the sequence length, and k is a number of mismatches.

8. The method of claim 7, wherein determining the second score comprises adding 2.2 times a second bit score to the second alignment score, wherein:
$$bit score = {\sum }_{l = 0}^{k} ( \begin{matrix}n \\ l\end{matrix} ) * \frac{{3}^{l}}{{4}^{n}}$$
where n is the sequence length, and k is a number of mismatches.

9. (canceled)

10. A computer-readable medium in which a program is stored for causing a computer to perform a method for identifying one or more D gene segment in a VDJ or VDDJ sequence, comprising:
obtaining a B cell receptor and/or T cell receptor data set, wherein the data set comprises a VDJ sequence;
aligning the VDJ sequence to one or more VDJ reference sequences, thereby generating a first potential alignment and a second potential alignment;
determining a first score for the first potential alignment and a second score for the second potential alignment in accordance with a D gene segment alignment scoring schema; and
identifying a D gene segment region associated with a highest score between the first score and the second score.

11. The computer-readable medium of claim 10, wherein aligning the VDJ sequence to one or more VDJ reference sequences comprises applying a first affine gap penalty function when aligning regions between VDJ segments of the VDJ sequence and a second affine gap penalty function when aligning other regions of the VDJ sequence.

12. The computer-readable medium of claim 11, wherein the first affine gap penalty function penalizes gap opens for insertion between VDJ segments at a first rate, and wherein the second affine gap penalty function penalizes gap opens for deletion bridging VDJ segments at a second rate, or penalizes other gap opens at a third rate that is larger than the first rate and the second rate, penalizes gap extends for insertion between VDJ segments at a fourth rate, and penalizes other gap extends at a fifth rate that is higher than the fourth rate.

13. The computer-readable medium of claim 10, further comprising:
applying a pre-determined scoring adjustment factor to the score of the 1^{st} and 2^{nd} potential alignments of the D gene segment region for the VDJ sequence; and/or
identifying the potential alignment with the highest score as a correct alignment of the D gene segment region; and/or
identifying an additional D gene segment, which is present in a VDDJ sequence.

14. (canceled)

15. The computer-readable medium of claim 10, wherein the scoring schema adds points to the score for each base match of a potential alignment of the D gene segment region to the reference VDJ sequence.

16. The computer-readable medium of claim 10, wherein:
the scoring schema subtracts points from the score for each base mismatch of a potential alignment of the D gene segment region to the reference VDJ sequence; and/or
the scoring schema subtracts points from the score for each gap that has to be opened for insertions in between V and D sequences and D and J sequences of a potential alignment of the D gene segment region to the reference VDJ sequence; and/or
the scoring schema subtracts points from the score for each gap that has to be deleted to close the gap between V and D sequences and D and J sequences of a potential alignment of the D gene segment region to the reference VDJ sequence; and/or
the scoring schema subtracts points from the score for all gap openings outside of the V-D-J junction of a potential alignment of the D gene segment region to the reference VDJ sequence; and/or
the scoring schema subtracts points from the score for all other gap extensions of a potential alignment of the D gene segment region to the reference VDJ sequence.

17. (canceled)

18. The computer-readable medium of claim 16, wherein the scoring schema subtracts points from the score for each gap extension in between the V and D sequences and D and J sequences of a potential alignment of the D gene segment region to the reference VDJ sequence.

19. (canceled)

20. (canceled)

21. (canceled)

22. (canceled)

23. A system for identifying one or more D gene segment in a VDJ or VDDJ sequence, the system comprising:
a data source configured to obtain a B cell receptor and/or T cell receptor data set, wherein the data set comprises a VDJ sequence, and
a processing unit configured to receive the B cell receptor and/or T cell receptor data set from the data source, the processing unit comprising:
an alignment engine configured to align the VDJ sequence to one or more VDJ reference sequences, thereby generating a first potential alignment and a second potential alignment;
a scoring engine configured to determine a first score for the first potential alignment and a second score for the second potential alignment in accordance with a D gene segment alignment scoring schema; and
an identification engine configured to identify a D gene segment region associated with a highest score between the first score and the second score.

24. The system of claim 23, the alignment engine further configured to align the VDJ sequence to one or more VDJ reference sequences comprising applying a first affine gap penalty function when aligning regions between VDJ segments of the VDJ sequence and a second affine gap penalty function when aligning other regions of the VDJ sequence.

25. (canceled)

26. The system of claim 23, the scoring engine further configured to:
apply a pre-determined scoring adjustment factor to the score of the 1^{st} and 2^{nd} potential alignments of the D gene segment region for the VDJ sequence; and/or
identify the potential alignment with the highest score as a correct alignment of the D gene segment region.

27. (canceled)

28. The system of claim 23, wherein the scoring schema adds points to the score for each base match of a potential alignment of the D gene segment region to the reference VDJ sequence.

29. The system of claim 23, wherein:
the scoring schema subtracts points from the score for each base mismatch of a potential alignment of the D gene segment region to the reference VDJ sequence; and/or
the scoring schema subtracts points from the score for each gap that has to be opened for insertions in between V and D sequences and D and J sequences of a potential alignment of the D gene segment region to the reference VDJ sequence; and/or
the scoring schema subtracts points from the score for each gap that has to be deleted to close the gap between V and D sequences and D and J sequences of a potential alignment of the D gene segment region to the reference VDJ sequence; and/or
the scoring schema subtracts points from the score for all gap openings outside of the V-D-J junction of a potential alignment of the D gene segment region to the reference VDJ sequence; and/or
the scoring schema subtracts points from the score for all other gap extensions of a potential alignment of the D gene segment region to the reference VDJ sequence.

30. (canceled)

31. The system of claim 29, wherein the scoring schema subtracts points from the score for each gap extension in between the V and D sequences and D and J sequences of a potential alignment of the D gene segment region to the reference VDJ sequence.

32. (canceled)

33. (canceled)

34. (canceled)

35. The system of claim 23, wherein the identification engine is further configured to identify an additional D gene segment, which is present in a VDDJ sequence.
